(19) United States
(12) Patent Application Publication
(10) Pub. No.: US 2014/0004159 A1
(43) Pub. Date: Jan. 2, 2014

(54) NANOFIBER SCAFFOLDS AND METHODS FOR REPAIRING SKIN DAMAGE

(71) Applicant: Marshall University Research Corporation, Huntington, WV (US)

(72) Inventors: Jingwei Xie, Chesapeake, OH (US); Bing Ma, Huntington, WV (US)

(21) Appl. No.: 13/931,250

(22) Filed: Jun. 28, 2013

Related U.S. Application Data
(60) Provisional application No. 61/666,260, filed on Jun. 29, 2012.

(51) Int. Cl. A61L 27/00; A61L 27/36; A61L 27/54

(52) U.S. Cl. CPC A61L 27/00; A61L 27/54; A61L 27/362
USPC 424/400; 424/93.7; 424/572; 264/484

(57) ABSTRACT

A composition is provided that includes a plurality of layered nanofiber scaffolds. A first nanofiber scaffold can include microwells configured to be seeded with one or more relevant cells, a skin tissue, or combinations thereof. Furthermore, the first nanofiber scaffold can comprise uniaxially aligned nanofibers between the microwells and random nanofibers on the microwells. The composite can also include a second nanofiber scaffold that comprises radially-aligned nanofibers. Further provided are methods for making such a composition as well as methods for treating damaged skin that include applying an effective amount of the composition to a site of damaged skin on a subject.

### RELATED APPLICATION

This application claims priority from U.S. Provisional Application Ser. No. 61/666,260, filed Jun. 29, 2012, the entire disclosure of which is incorporated herein by this reference.

### TECHNICAL FIELD

The presently-disclosed subject matter relates to nanofiber scaffolds and methods of using the scaffolds for repairing skin damage. In particular, the presently-disclosed subject matter relates to nanofiber scaffolds including arrayed microwells and structural cues to mimic the hierarchical architecture of the extracellular matrix that is present in the dermis, and to provide a scaffold for seeding one or more relevant cells and/or skin tissue to thereby repair skin damage.

### BACKGROUND

Accidents, war, and terrorist attacks often cause skin damage to subjects over a large area. Also, aging, diabetes and chronic vascular diseases lead to hard-to-heal wounds that have presented significant clinical challenges. Furthermore, burn injuries have increased with not only the development of modern weapons, but also in ordinary life, where 450,000 burn injuries receive medical treatment every year in the United States alone. Other causes of skin damage include trauma and chronic ulcerations secondary to diabetes mellitus, pressure, and venous stasis. Indeed, in a recent fact sheet published by the Center for Disease Control and Prevention, the estimated prevalence of diabetes in the US population was 7% or 21 million individuals, and up to 10% of these individuals (2 million) had chronic diabetic ulcers, many of which (approximately 82,000) eventually necessitated amputation.

Split-skin grafts used to treat full thickness skin loss in burns are a gold-standard treatment; however, they lead to scar formation that is often vulnerable and unstable in the donor site. Currently, the most common clinical treatments for skin transplantation include postage stamp skin grafting, mesh skin grafting, MEEK skin grafting, and allo- and auto-mixed skin grafting. The basic principle shared by all these methods is to cover as great a wound area as possible with islands of transplanted skin that are separated by a distance. For instance, the maximum distance is 6 mm between transplanted skin tissues for MEEK skin grafts (skin tissue 3 mm×3 mm, expansion ratio: 1:9). The wound healing process comprises the formation of skin islands from transplanted skin tissues, the subsequent expansion of the skin inlands into the surrounding areas, and, finally, coverage of whole wound area.

However, issues still remain unresolved for skin grafting. For example, skin grafting lacks approaches to promote skin regeneration between the transplanted skin islands, and may require additional surgeries for residual wound areas or to keep the distance between the transplanted skin islands consistent. Moreover, skin grafting lacks higher expansion ratios for skin transplantation, and may form hypertrophic scar tissue that is itchy, painful, hard, and unsightly. In addition, donor skin that is too thick is not suitable for MEEK transplantation and affects the survival of transplanted skin islands, and curled transplanted skin on the wound area can cause transplantation failure. Current skin grafts are also relatively expensive, labor intensive, and complex to implement. Additionally, patients with chronic diseases cannot endure large operations and anesthesia because the function of their organs is damaged, and these patients can only rely on simple surgical dress changes to treat wound areas.

In this regard, many engineered skin products have been used clinically, such as epidermis substitutes (i.e., Laserskin, CellSpray, BioSeed-S, LyphoDerm), dermis substitutes (i.e., acellular: Integra, AlloDerm, Biobrane; cellular: Transcyte, Dermagraft), and composite allografts. However, skin cell culture still requires harvesting skin (e.g., 25 cm²) from patients and culture for 2-5 weeks. Hence, it is a lab-intensive, complicated, and costly procedure. The use of culture medium may also affect clinical safety, and the quality of skin at a wound site after healing is usually poor and blisters often occur. For instance, a burn that appears superficially may deepen over a period of 48 to 72 hours, with the zone of stasis becoming necrotic. These factors limit the application of current engineered skin in clinical settings.

With regard to repairing skin damage, a concept for tissue engineering is to combine tissue engineering strategies by making use of both biomaterials, cells, and/or growth factors and micrografts, such as split thickness skin grafts. However, current designs still fail to meet long-felt but unmet needs, and still require minimal skin tissue harvesting to cover a wound area (e.g., large expansion ratio), constant distances between transplanted skin islands, and wound healing consistency (e.g., arrayed skin islands). Known technologies strive to, but fall short of, being simple to implement, having a high survival rate, accelerating epithelialization of the wound area among the transplanted skin islands, being biologically safe, and being low cost. Thus, a scaffold that meets these needs and can aid in the repair of damaged skin tissue would be highly desirable and beneficial.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes compositions that comprise one or more nanofiber scaffolds. In an exemplary embodiment, the composition comprises: a first nanofiber scaffold including microwells configured to be seeded with one or more relevant cells, a skin tissue, or combinations thereof; and a second nanofiber scaffold layered on the first nanofiber scaffold. In some embodiments, the microwells are dimensioned to have a diameter of about 0.1 mm to about 10 mm and/or a depth of about 20 μm to about 2 mm. In some embodiments the microwells are arranged in a square array, a hexagonal array, or a combination thereof.

In some embodiments, the nanofibers of the nanofiber scaffolds are aligned in a particular arrangement. For instance, in some embodiments the first nanofiber scaffold comprises uniaxially-aligned nanofibers between the microwells and random nanofibers on the microwells. Also, in some embodiments the second nanofiber scaffold can comprise radially-aligned nanofibers.

The nanofibers of the composition can be comprised of a biodegradable polymer. Exemplary biodegradable polymers include those selected from the group consisting of synthetic polymers, natural polymers, inorganic materials, and combinations thereof. In certain embodiments the biodegradable polymer is polycaprolactone.

Furthermore, and as mentioned above, the first nanofiber scaffold of the composition can be configured to be seeded with one or more relevant cells, a skin tissue, or combinations thereof. In some embodiments, the relevant cells are selected from the group consisting of adult stem cells, embryonic stem cells, induced pluripotent cells, primary skills, and combinations thereof. In some embodiments, the adult stem cells are adipose-derived stem cells, and in other embodiments, the primary cells are skin cells. The provided skin tissue can be skin tissue that has been minced. In some embodiments, skin tissue is minced so as to have a size of about 1 mm³ or less. In some embodiments, each piece of skin tissue is about 0.1 mm to about 1.0 mm in diameter.

Embodiments of the composition can further comprise various additional agents, which optionally can be attached to the first nanofiber scaffold, the second nanofiber scaffold, or both. For instance, some embodiments of compositions further comprise a growth factor, such as a vascular endothelial growth factor (VEGF), a basic fibroblast growth factor (bFGF), an insulin-like growth factor (IGF), a placental growth factor (PIGF), Ang1, a platelet derived growth factor-BB (PDGF-BB), a transforming growth factor β (TGF-β), human epidermal growth factor (hEGF), keratinocyte growth factor, and combinations thereof. In some embodiments, the composition can comprise a therapeutic agent, such as an anti-inflammatory agent, an antibiotic, or a combination thereof. Exemplary compositions can also comprise an extracellular matrix protein, where, in some embodiments, the extracellular matrix protein can be, but is not limited to, fibronectin, laminin, collagen, or a combination thereof.

The presently-disclosed subject matter also includes methods for treating damaged skin in a subject. In some embodiments, a method for treating damaged skin comprises a first step of providing a first nanofiber scaffold including microwells seeded with one or more relevant cells, a skin tissue, or combinations thereof, and a second nanofiber scaffold layered on the first nanofiber scaffold. Subsequently, the method comprises a second step of applying an effective amount of the composition to a site of damaged skin on the subject. In some embodiments, the effective amount of the composition is an amount of composition sufficient to cover at least the damaged skin.

Further still, the presently-disclosed subject matter includes methods for making the nanofiber scaffold compositions. In some embodiments, the method comprises a first step of electrospinning a first biodegradable polymer onto a first collector comprising beads to create a first nanofiber scaffold that includes microwells configured to be seeded with one or more relevant cells, a skin tissue, or combinations thereof. The method then comprises a second step of electrospinning a second biodegradable polymer onto a second collector comprising a ring electrode and a point electrode to create a second nanofiber scaffold. Subsequently, in some embodiments, the method comprises a third step of seeding the one or more relevant cells, the skin tissue, or combinations thereof in the microwells of the first nanofiber scaffold. Lastly, the exemplary method comprises a fourths step of layering the second nanofiber scaffold on the first nanofiber scaffold. In some embodiments, the beads that comprise the first collector can have a diameter of about 0.1 mm to about 10 mm. In some embodiments, the beads that comprise the first collector can be arranged in a square array, a hexagonal array, or combinations thereof. The beads can be stainless steel beads or the like.

Further features and advantages of the present invention will become evident to those of ordinary skill in the art after a study of the description, figures, and non-limiting examples in this document.

### BRIEF DESCRIPTION OF THE DRAWINGS

FIGS. 1A-1D include a schematic diagram of an electrospinning setup for generating nanofiber scaffolds having radially-aligned nanofibers (FIG. 1A), a schematic diagram of the electric field of strength vectors for the region between a spinneret and a collector (FIG. 1B), and a photograph (FIG. 1C) and a scanning electron microscopy (SEM) image (FIG. 1D) showing a nanofiber scaffold having radially-aligned nanofibers;

FIGS. 2A-2D include fluorescence micrographs showing dura tissues that were cultured for 4 days on embodiments of nanofiber scaffolds having radially-aligned (FIGS. 2A and 2C) and random nanofibers (FIGS. 2B and 2D), where the dashed circle line indicates the border of dura cells after seeding at day 0, and where arrows mark the center of the scaffold in the magnified views of FIG. 2C and FIG. 2D of the center portions shown in FIG. 2A and FIG. 2B, respectively;

FIGS. 3A-3D include fluorescence micrographs showing the migration of dura fibroblasts seeded for 1 day on embodiments of nanofiber scaffolds that were radially-aligned and bare (FIG. 3A), random and bare (FIG. 3B), radially-aligned with a fibronectin coating (FIG. 3C), and random with a fibronectin coating (FIG. 3D);

FIGS. 4A-4F include fluorescence micrographs showing migration of dura fibroblasts seeded on fibronectin coated scaffolds of radially-aligned nanofibers after 1 day (FIG. 4A), 3 days (FIG. 4B), and 7 days (FIG. 4C) (higher magnification shown in FIG. 4D), and further includes an illustration with a dashed line that indicates a void space used for calculations (FIG. 4E) as well as a graph showing the void space area as a function of incubation time for four embodiments of scaffolds (* and # indicate p<0.05) (FIG. 4F);

FIGS. 5A-5B include schematics showing an electrospinning setup for fabricating a nanofiber membrane with microwells and structural cues therebetween (FIG. 5A), and electric field vectors in the region between the electrospinning jet (needle) and the stainless steel beads (collector) (FIG. 5B);

FIGS. 6A-6F include images showing different embodiments of nanofiber scaffolds composed of electrospun polycaprolactone (PCL) nanofibers that were taken using optical microscopy with light exposure from the right-hand side (FIG. 6A) and SEM (FIGS. 6B-6F);

FIGS. 7A-7D include SEM images showing the convex side of embodiments nanofiber membranes fabricated using collectors that included hexagonal arrays of stainless steel beads with different distances between neighboring beads (FIGS. 7A-7B), a square array of stainless steel beads (FIG. 7C), and a square array of stainless steel beads with a gradual increase in distances between adjacent beads (FIG. 7D);

FIGS. 8A-8D include an optical microscopy image showing MG-63 cell-containing droplets seeded in the microwells of an embodiment of a nanofiber scaffold (FIG. 8A), and further include fluorescence micrographs showing microarrays of live MG63 cells stained with fluorescein diacetate having about 50 cells per well after incubation for 1 day (FIG. 8B), about 150 cells per well after incubation for 1 day (FIG. 8C), and about 150 cells per well after incubation for 3 days (FIG. 8D);

FIGS. 9A-9B include SEM images showing an embodiment of a nanofiber membrane composed of electrospun PCL nanofibers (FIG. 9A), and further includes images showing the specific regions (FIGS. 9B-9D) indicated in FIG. 9A;

FIGS. 10A-10B include fluorescent images showing migration of MG63 cells seeded to an embodiment of a nanofiber scaffold with a hexagonal array of microwells taken at low magnification (FIG. 10A) and high magnification (FIG. 10B);

FIG. 11 includes a schematic showing a collector made of arrayed pins capped with metal balls for generating nanofiber scaffolds with a distance between arrayed microwells;

FIGS. 12A-12D include schematic diagrams showing a nanofiber scaffold with arrayed microwells and structural cues (FIG. 12A), minced skin tissues seeded in microwells of the nanofiber scaffold (FIG. 12B), a radially-aligned nanofiber scaffold (FIG. 12C), and a “sandwich” structure formed by layering radially-aligned nanofiber scaffold(s) on the scaffold seeded with minced skin tissue (FIG. 12D);

FIGS. 13A-13B include fluorescent images showing minced embryo chick skin tissues seeded in microwells of a nanofiber scaffold for 4 days alone (FIG. 13A) and with a radially-aligned nanofiber scaffold layered thereon (FIG. 13B);

FIGS. 14A-14D include images showing steps in a procedure for transplanting a nanofiber skin scaffold that comprise forming an excision consisting of a 2 cm in diameter skin defect (FIG. 14A), and covering the skin defect covered with the nanofiber scaffold (FIG. 14B), suturing (FIG. 14C), and covering with gauze (FIG. 14D);

FIG. 15 includes an image showing two skin defects after a three week treatment with nanofiber scaffolds comprising minced skin tissue (left) and nanofiber scaffolds alone (right);

FIGS. 16A-16B include schematic diagrams illustrating the electrospinning setup for fabricating nanofiber membranes with squared arrayed microwells and structural cues on the surface (FIG. 16A), and further illustrating the electric field vectors and streamlines in the region between the needle and the collector (FIG. 16B);

FIGS. 17A-17D include images of PCL nanofiber membranes with square arrayed microwells and structural cues, including optical micrograph (FIG. 17A) and SEM images (FIGS. 17B-17D) of a PCL nanofiber membrane with square arrayed microwells and structural cues, where FIGS. 17C-17D are magnified views of regions C and D in FIG. 17B, and where the distance between the two adjacent microwells was 3 mm;

FIG. 18 includes images showing the results of experiments where different numbers of NIH 3T3 fibroblasts were seeded to each microwell of nanofiber membranes and incubated for 3, 7, 14 and 21 days;

FIG. 19 includes images showing how the distance between two adjacent microwells affects cell coverage on the nanofiber membrane subsequent to seeding one hundred NIH 3T3 fibroblasts to each microwell of nanofiber membranes with distances between two adjacent wells of 2 and 6 mm and incubated for 3, 7, 14 and 21 days;

FIG. 20 includes images showing radially-aligned nanofibers promoting the migration of cells seeded to nanofiber microwell membranes, where radially-aligned nanofibers were laid on the top of nanofiber membranes immediately after seeding of 100 NIH 3T3 cells to each microwell forming a sandwich-type nanofiber scaffold which was incubated for 3, 7, 14 and 21 days, and where the distance between two adjacent microwells was 3 mm;

FIG. 21 includes images showing that primary rat skin cells show similar behavior to NIH 3T3 fibroblasts when cultured in the microwells of nanofiber membranes, where one hundred primary rat skin cells were seeded to each microwell of nanofiber membranes and incubated for 3, 7, 14 and 21 days, and where the distance between two adjacent microwells was 3 mm;

FIGS. 22A-22B include graphs showing cell migration quantified by measuring area fractions occupied by cells on nanofiber scaffolds under different conditions (FIG. 22A; 10-3T3-M: 10 NIH 3T3 fibroblasts were seeded into each microwell of nanofiber membranes; 100-3T3-M: 100 NIH 3T3 fibroblasts were seeded into each microwell of nanofiber membranes; 1000-3T3-M: 1000 NIH 3T3 fibroblasts were seeded into each microwell of nanofiber membranes; 100-3T3-M+R: 100 NIH 3T3 fibroblasts were seeded into each microwell of nanofiber membranes and covered by radially-aligned nanofibers; 100-Skin cells-M: 100 primary skin cells were seeded into each microwell of nanofiber membranes) and showing distances between two adjacent microwells affected the area fraction (FIG. 22B).

FIGS. 23A-23D are images of micrographs showing wounds on day 0 (FIG. 23A), 7 (FIG. 23B), 14 (FIG. 23C) and 21 (FIG. 23D) after implantation of sandwich-type nanofiber scaffolds (left) and petrolatum gauze only (right), where a one 2-cm diameter circular full-thickness skin excision wound was created on each side of the dorsal surface (FIG. 23A), where nanofiber scaffolds and microskins indicated by an arrow head were evenly distributed and adhered tightly to the wound on day 7 post-surgery (FIG. 23B), where nanofiber scaffolds and microskins indicated by an arrow head adhered tightly to wound bed, allowing drainage to pass through and adhere to gauze dressing on day 14 post-surgery (FIG. 23C); and where nanofiber scaffolds and microskins attached well on wound bed even significant wound contraction occurred on day 21 post-surgery indicated by an arrow head (FIG. 23D);

FIGS. 24A-24G are images showing representative hematoxylin and eosin staining of skin tissue sections illustrating the healing process of wounds after surgical application of sandwich-type nanofiber scaffolds at day 7 (FIG. 24A), 14 (FIG. 24B) and 21 (FIG. 24C), where the black arrowheads in these images indicate the boundaries between wound and surrounding normal skin, where transplanted microskins indicated by small black arrows in sandwich-type nanofiber scaffolds ‘took’ satisfactorily on wounds with a uniform distribution at day 7 post-surgery (FIG. 24A); where re-epithelialization derived from microskin occurred along the wound bed at day 14 after surgery (FIG. 24B), where the wound was completely closed by re-epithelialization derived from microskins indicated by black arrows at day 21 after surgery (FIG. 24C), where a magnified view of the region D in FIG. 24A shows that the microskin contained both epidermal layer and dermal layer indicated by white dash lines and white arrow heads, respectively, which was confined by the nanofiber microwell indicated by black dash lines (FIG. 24D), where a magnified view of the region E in FIG. 24D shows small blood vessels indicated by white arrow heads, large collagen bundles and few fibroblasts in dermal layer of microskin (FIG. 24E), where a magnified view of the region F in FIG. 24B shows that stratified epithelial cells derived from microskins crept along the surface of wound bed towards the adjacent microskin indicated by white dash lines and simultaneously the dermal layer of microskins began integrating with the wound bed indicated by white arrow heads (FIG. 24F), and where a magnified view of the region G in FIG. 24C shows epidermal cells migrated from the two adjacent microskins resurfaced the wound indicated by white dash lines (FIG. 24G);

FIGS. 25A-25G are images showing representative hematoxylin and eosin staining of skin tissue sections illustrating healing process of wound treated with petrolatum gauzes at day 7 (FIG. 25A), 14 (FIG. 25B), and 21 days (FIG. 25C) post-surgery, where black arrowheads in the images indicated the boundaries between wound and surrounding normal skin, where epithelial cells migrated from edges of normal skin toward the center of wound (FIG. 25A), where epithelial cells were not found on wound bed on day 14 post-surgery (FIG. 25B), where a big wound gap still existed on day 21 post-surgery (FIG. 25C), where a magnified view of the region D in FIG. 25A demonstrated small blood vessels and fibroblasts grew from the bottom of wound bed and that the wound was repaired by fresh granulation tissue (FIG. 25D), where a magnified view of the regions E in FIG. 25B show that epithelial cells derived from normal skin migrated along wound bed to repair the wound indicated by white dash lines and the head of epithelial cell sheet crept on the granulation tissue containing small vessels and fibroblasts (FIG. 25E), where a magnified view of the region F in FIG. 25B shows small vessels and fibroblasts on the wound bed (FIG. 25F), and where a magnified view of the region G in FIG. 25C shows that the wound was repaired by granulation tissue and the occurrence of a dramatic decrease of numbers of small vessels and fibroblasts and a significant increase of collagen content in granulation tissue (FIG. 25G);

FIG. 26 includes images showing immunohistochemistry performed on skin tissue sections after sandwich-type nanofiber skin graft treatment for 7 (upper images), 14 (middle images), and 21 days (lower images);

FIG. 27 includes images showing immunohistochemistry performed on skin tissue sections after petrolatum gauzes treatment for (7 (upper images), 14 (middle images), and 21 days (lower images);

FIG. 28 includes images showing cross sections of microwell nanofiber membranes embedded in PDMS illustrating the depth and diameter of microwells;

FIG. 29 includes photographs showing nanofiber membranes with different distances between two adjacent microwells and diameters of microwells, where the distances between two adjacent metal beads were (A) 1.5 mm, (B) 3 mm, (C) 6 mm and (D) 6 mm, and where the diameters of the metal beads were (A-C) 1.5 and (D) 3 mm; and

FIG. 30 includes images of NIH 3T3 fibroblasts cultured on different PCL nanofiber assemblies, including random nanofibers (top panels), uniaxially-aligned nanofibers (middle panels), and nanofiber scaffolds of the presently-disclosed subject matter having arrayed microwells and structural cues (bottom panels) for 14 days.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by one of ordinary skill in the art, definitions are set forth to facilitate explanation of the presently-disclosed subject matter. Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The presently-disclosed subject matter includes nanofiber scaffolds and methods of using the scaffolds for repairing and regenerating damaged skin tissue. In particular, in some embodiments, the presently-disclosed subject matter relates to nanofiber scaffolds with arranged microwells and structural cues to mimic the hierarchical architecture of the extracellular matrix that is present in the dermis, and to provide a scaffold for seeding one or more relevant cells and/or skin tissue to thereby repair skin damage.

The term “nanofiber” is used herein to refer to materials that are in the form of continuous filaments or discrete elongated pieces of material, and that typically have diameters of less than or equal to 1000 nanometers. In this regard, the term “nanofiber scaffold” is used herein to refer to the arrangement of such nanofibers into a supporting framework that can then be used to support cells or other additional materials. Various methods known to those of ordinary skill in the art can be used to produce nanofibers, including, but not limited to, interfacial polymerization and electrospinning. For example, in some embodiments, electrospinning techniques can be used to generate nanofibers from a variety of materials, including polymers, composites, and ceramics. Typically, such electrospinning techniques make use of a high-voltage power supply, a spinneret (e.g., a hypodermic needle), and an electrically conductive collector (e.g., aluminum foil). To perform the electrospinning process using these materials, an electrospinning liquid (i.e., a melt or solution of the desired materials that will be used to form the nanofibers) is generally first loaded into a syringe and is then fed at a specific rate set by a syringe pump. In some cases, a well-controlled environment (e.g., humidity, temperature, and atmosphere) can be used to achieve a smooth, reproducible operation of electrospinning

As the liquid is fed by the syringe pump with a sufficiently high voltage, the repulsion between the charges immobilized on the surface of the resulting liquid droplet overcomes the confinement of surface tension and induces the ejection of a liquid jet from the orifice. The charged jet then goes through a whipping and stretching process, and subsequently results in the formation of uniform nanofibers. Further, as the jet is stretched and the solvent is evaporated, the diameters of the fibers can then be continuously reduced to a scale as small as tens of nanometers and, under the influence of an electrical field, the nanofibers can subsequently be forced to travel towards a grounded collector, onto which they are typically deposited as a non-woven mat. In some embodiments, due to the high ratio of surface area to volume and the one-dimensional morphology, electrospun nanofibers can mimic the architecture of the extracellular matrix.

In some embodiments the nanofiber scaffolds can be created to include structural cues, such as uniaxially-aligned, orthogonally-crossed, randomly -aligned, and radially-aligned nanofibers. These structural cues can be formed by manipulating the electrical field and/or using mechanical force during electrospinning. For example, in some embodiments of the presently-disclosed subject matter, the collector to which the nanofibers travel comprises a ring electrode, which can be a metal ring, and a point electrode located within the ring electrode, which can be a sharp needle. Nanofibers deposited on collectors comprising a ring electrode and a point electrode form nanofiber scaffolds having radially-aligned nanofibers.

Furthermore, in some embodiments, the nanofibers themselves can include various secondary structures, including, but not limited to, microwells, core-sheath structures, hollow structures, porous structures, and the like. In this regard, the term “microwell” is used herein to refer to an indentation, recess, cavity, or the like on a nanofiber scaffold that is configured to be seeded with one or more relevant cells, a skin tissue, or any other liquid or solid substance. Of course, embodiments of nanofiber scaffolds described herein can comprise any desired number and density of microwells, and can comprise, in some embodiments, about 10, about 20, about 30, about 40, about 50, about 60, about 70, about 80, about 90, about 100, about 200, about 300, about 400, about 500, about 600, about 700, about 800, about 900, or about 1000 microwells.

In some embodiments, the microwells are formed by electrospinning the nanofibers onto a collector that comprises beads. The nanofibers deposited on such a collector can conform to the shape of the beads, and the beads' exterior surfaces largely define the shape and dimension of the microwells. Additionally, the resulting microwells can be formed in any arrangement by modifying, among other things, the arrangement of beads in the collector. For example, in some embodiments the beads are configured so that the resulting microwells are arranged in a square array, a hexagonal array, or combinations thereof. In this regard, the term “bead” is used herein to refer to any object that has a dimension desired for a microwell, and in some embodiments, includes spherical stainless steel beads. While beads can be of any shape or size, in some embodiments, spherical beads are utilized that have a diameter of about 0.1 mm, about 0.2 mm, about 0.3 mm, about 0.4 mm, 0.5 mm, 0.6 mm, 0.7 mm, 0.8 mm, 0.9 mm, about 1 mm, about 2 mm, about 3 mm, about 4 mm, about 5 mm, about 6 mm, about 7 mm, about 8 mm, about 9 mm, or about 10 mm. Furthermore, the beads can be comprised of any material, but are generally comprised of a conductive material when used in conjunction with electrospinning techniques or the like.

In some embodiments of the presently-disclosed subject matter, the nanofibers that are electrospun are comprised of a biodegradable polymer. The term “biodegradable” as used herein is intended to describe materials that degrade under physiological conditions to form a product that can be metabolized or excreted without damage to the subject. In certain embodiments, the product is metabolized or excreted without permanent damage to the subject. Biodegradable materials may be hydrolytically degradable, may require cellular and/or enzymatic action to fully degrade, or both. Biodegradable materials also include materials that are broken down within cells. Degradation may occur by hydrolysis, oxidation, enzymatic processes, phagocytosis, or other processes.

Such biodegradable polymers are known to those of ordinary skill in the art and include, but are not limited to, synthetic polymers, natural polymers, blends of synthetic and natural polymers, inorganic materials, and the like. In some embodiments, the nanofibers are comprised of polycaprolactone. In some embodiments, blends of polymers are utilized to form the nanofibers to improve their biocompatibility as well as their mechanical, physical, and chemical properties. Regardless of the particular polymer used to produce the nanofibers, once the nanofibers have been created by the electrospinning process, the nanofibers are subsequently assembled into a nanofiber scaffold. Numerous methods of assembling nanofiber scaffolds can, of course, be used in accordance with the presently-disclosed subject matter.

In some embodiments, the materials used to produce the nanofibers are selected from those listed in Table 1 below. See also, e.g., Xie J. et al. Macromolecular Rapid Communications, 2008, 29, 1775, which is incorporated herein by reference in its entirety.

TABLE 1
Exemplary Materials for Electrospinning.

| Materials^{a)} | Solvent |
|---|---|
| Natural polymers | |
| Chitosan | 90% Acetic acid |
| Gelatin | Formic acid |
| Gelatin | TFE |
| Collagen Type I, II, and III | HFIP |
| Collagen Type I, II, and III | HFIP |
| Collagen Type I, II, and III | HFIP |
| Elastin | HFIP |
| Hyaluronic acid | DMF/water |
| Cellulose | NMMO/water |
| Silk fibroin | Methanol |
| Phospholipids (Lecithin) | Chloroform/DMF |
| Fibrinogen | HFIP/10 x minimal essential medium |
| Hemoglobin | TFE |
| Fibrous calf thymus Na-DNA | Water/ethanol |
| Virus M13 viruses | THF |
| Synthetic polymers | |
| PLGA | TFE/DMF |
| PLA | HFIP |
| PLA | DCM |
| PLA | DCM/DMF |
| PLA | DCM/pyridine |
| PCL | DCM/methanol |
| PHBV | Chloroform/DMF |
| PDO | HFIP |
| PGA | HFIP |
| PLCL | Acetone |
| PLCL | DCM |
| PLLA-DLA | Chloroform |
| PEUU | HFIP |
| Cellulose acetate | Acetic acid/water |
| PEG-b-PLA | Chloroform |
| EVOH | 70% propan-2-ol/water |
| PVA | Water |
| PEO | Water |
| PVP | Ethanol/water |
| Blended | |
| PLA/PCL | Chloroform |
| Gelatin/PVA | Formic acid |
| PCL/collagen | HFIP |
| Sodium aliginate/PEO | Water |
| Chitosan/PEO | Acetic acid/DMSO |
| Chitosan/PVA | Acetic acid |
| Gelatin/elastin/PLGA | HFIP |
| Silk/PEO | Water |
| Silk fibroin/chitosan | Formic acid |
| PDO/elastin | HFIP |
| PHBV/collagen | HFIP |
| Hyaluronic acid/gelatin | DMF/water |
| Collagen/chondroitin sulfate | TFE/water |
| Collagen/chitosan | HFIP/TFA |
| Composites | |
| PDLA/HA | Chloroform |
| PCL/CaCO₃ | Chloroform/methanol |
| PCL/CaCO₃ | DCM/DMF |
| PCL/HA | DCM/DMF |
| PLLA/HA | Chloroform |
| Gelatin/HA | HFIP |
| PCL/collagen/HA | HFIP |
| Collagen/HA | HFIP |
| Gelatin/siloxane | Acetic acid/ethyl acetate/water |
| PLLA/MWNTs/HA | 1,4-dioxane/DCM |
| PLGA/HA | DCM/water |

Regardless of the particular polymer used to produce the nanofibers, once the desired configuration of the nanofiber scaffold(s) has been produced, in some embodiments the nanofiber scaffold is then seeded with one or more relevant cells, a skin tissue, or combinations thereof, as it has been discovered that the nanofiber scaffolds of the presently-disclosed subject matter provide favorable conditions for the relevant cells and/or skin tissue to adhere, proliferate, and organize. Typically, the cells or skin tissues can be seeded onto the nanofiber scaffold in any manner known to those of ordinary skill in the art.

In some embodiments, however, the relevant cells are seeded onto the scaffolds by first forming a solution of medium and relevant cells, and then loading a droplet of that solution directly into the microwells of the nanofiber scaffold. Without being bound by theory or mechanism, it is believed that such a depositing of cells is particularly beneficial as it allows the cell-laden droplet to maintain its spherical shape due to the geometric confinement of the cells and the surface hydrophobicity of the nanofibers in the scaffold. Using scaffolds that are seeded with relevant cells is also beneficial because they can eliminate the need to procure skin grafts.

With further regard to the relevant cells that are, in some embodiments, seeded into the microwells of the nanofiber scaffolds, the term “relevant cells,” is used to refer to cells that are appropriate for incorporation into a nanofiber scaffold of the presently-disclosed subject matter, based on the intended use of that scaffold. For example, relevant cells that are appropriate for the repair, restructuring, or repopulation of particular damaged tissue or organ will typically include cells that are commonly found in that tissue or organ or that can give rise to cells that are commonly found in that tissue or organ by differentiation or some other mechanism of action. In that regard, exemplary relevant cells that can be incorporated into tissue constructs of the presently-disclosed subject matter include stem cells, skin cells, neurons, cardiomyocytes, myocytes, chondrocytes, pancreatic acinar cells, islets of Langerhans, osteocytes, hepatocytes, Kupffer cells, fibroblasts, keratinocytes, myoblasts, satellite cells, endothelial cells, adipocytes, preadipocytes, biliary epithelial cells, and the like. These types of cells may be isolated and cultured by conventional techniques known in the art. Exemplary techniques can be found in, among other places; Freshney, Culture of Animal Cells, A Manual of Basic Techniques, 4th ed., Wiley Liss, John Wiley & Sons, 2000; Basic Cell Culture: A Practical Approach, Davis, ed., Oxford University Press, 2002; Animal Cell Culture: A Practical Approach, Masters, ed., 2000; and U.S. Pat. Nos. 5,516,681 and 5,559,022.

As used herein, the term “stem cells” refers broadly to traditional stem cells, progenitor cells, preprogenitor cells, precursor cells, reserve cells, and the like. Exemplary stem cells include, but are not limited to, embryonic stem cells, adult stem cells, pluripotent stem cells, induced pluripotent stem cells, neural stem cells, liver stem cells, muscle stem cells, muscle precursor stem cells, endothelial progenitor cells, bone marrow stem cells, chondrogenic stem cells, lymphoid stem cells, mesenchymal stem cells, hematopoietic stem cells, central nervous system stem cells, peripheral nervous system stem cells, and the like. Descriptions of stem cells, including methods for isolating and culturing them, may be found in, among other places, Embryonic Stem Cells, Methods and Protocols, Turksen, ed., Humana Press, 2002; Weisman et al., Annu Rev. Cell. Dev. Biol. 17:387-403; Pittinger et al., Science, 284:143-47, 1999; Animal Cell Culture, Masters, ed., Oxford University Press, 2000; Jackson et al., PNAS 96(25):14482-86, 1999; Zuk et al., Tissue Engineering, 7:211-228, 2001; and U.S. Pat. Nos. 5,559,022, 5,672,346 and 5,827,735. One of ordinary skill in the art will understand that the stem cells that are selected for inclusion in a nanofiber scaffold are typically selected when such cells are appropriate for the intended use of a particular construct.

In some embodiments of the nanofiber scaffolds, the relevant cells that are seeded on the nanofiber scaffold are selected from the group consisting of adult stem cells, embryonic stem cells, induced pluripotent cells, or primary cells. In some embodiments, the relevant cells are adult stem cells. In some particular embodiments, the adult stem cells are adipose-derived stem cells, as such adipose-derived stem cells have been surprisingly found to be particularly useful in the nanofiber scaffolds of the presently-disclosed subject matter.

The term “skin tissue,” as used herein, refers to any tissue, including epidermis, dermis and basement membrane tissue, that is derived from the skin of a subject and is appropriate for incorporation into a nanofiber scaffold of the presently-disclosed subject matter, based on the intended use of that scaffold. In some embodiments, the skin tissue is either autograft, which is derived from the subject's own body, or allograft, which is derived from a genetically dissimilar member of the same species. In some cases though, the graft material can even be xenograft, which is taken from another species. In some embodiments, the skin tissue is minced. The term “minced” is used herein to refer to skin tissue that is divided into smaller-sized (e.g., approximately even-sized and smaller) pieces of any desired shape and size. In some embodiments of the present invention, the pieces of minced skin tissue are each about 1 mm³ and are thus approximately equal in length, width, and height. In some embodiments, each piece of skin tissue is about 0.1 mm to about 1.0 mm in diameter. As with seeded relevant cells, seeded skin tissue can provide, among other things, cells that will enhance and improve the ability of a nanofiber scaffold to repair damaged skin tissue. Since embodiments of the nanofiber scaffolds can be loaded with non-continuous and/or minced skin tissue, the scaffolds can eliminate the need to procure large skin grafts, which can be painful, unsightly, and difficult to obtain.

In this regard, some embodiments of nanofiber scaffolds have the added benefit of increasing the migration of cells, whether they are relevant cells, cells from skin tissue, or a subject's cells, in a manner that can enhance and improve the rate and quality of repair of damaged skin. For example, in some embodiments of nanofiber scaffolds that have radially-aligned nanofibers, cell migration from the periphery to the center of the nanofiber scaffold is enhanced. As a further example, in some embodiments that make use of a nanofiber scaffold including microwells that are seeded with one or more relevant cells and/or skin tissue, the nanofiber scaffold comprises uniaxially-aligned nanofibers between microwells and random nanofibers on the microwells. In such embodiments, the uniaxially-aligned nanofibers can enhance cell migration from the seeded microwells to the surrounding areas and the other microwells. Furthermore, it has been discovered that embodiments of nanofiber scaffolds including microwells can mimic the architecture of the extracellular matrix. These and other characteristics can further enhance the ability of nanofiber scaffolds to enhance and improve the repair of damaged skin.

In some embodiments, in addition to seeding (loading) the nanofiber scaffolds with one or more relevant cells and/or skin tissue, various additional materials and/or biological molecules can be attached to the nanofiber scaffolds. The term attached includes, but is not limited to, coating or incorporating by any means the additional materials and/or biological molecules, and attached can refer to incorporating such components on all the nanofiber scaffolds of a composition, fewer than all the nanofiber scaffolds in a composition, or only on a portion of one or more nanofiber scaffolds in a composition. For example, in some embodiments, to improve the adherence and incorporation of a nanofiber scaffold to a damaged tissue, an extracellular matrix protein, such as, in some embodiments, fibronectin, laminin, and/or collagen, is further attached to the nanofiber scaffold.

As another example of materials that can be attached to or used to coat the nanofiber scaffolds, in some embodiments, a growth factor is further attached to the nanofiber scaffold to facilitate the repair and regeneration of the damaged tissue. In some embodiments, the growth factor is selected from the group consisting of vascular endothelial growth factor (VEGF), basic fibroblast growth factor (bFGF), insulin-like growth factor (IGF), placental growth factor (PIGF), Ang1, platelet derived growth factor-BB (PDGF-BB), and transforming growth factor β (TGF-β), human epidermal growth factor (hEGF), keratinocyte growth factor, and combinations thereof. In some embodiments, the growth factor is VEGF. Of course, as would be recognized by those of ordinary skill in the art, various other materials and biological molecules can be attached to or used to coat a nanofiber scaffold of the presently-disclosed subject matter, and can be selected for a particular application based on the tissue to which they are to be applied.

In some embodiments of the presently-disclosed subject matter, a therapeutic agent (i.e., an agent capable of treating damaged tissue as defined herein) is further attached to the nanofiber scaffold. In some embodiments, the therapeutic agent is an anti-inflammatory agent or an antibiotic. Examples of anti-inflammatory agents that can be incorporated into the scaffolds include, but are not limited to, steroidal anti-inflammatory agents such as betamethasone, triamcinolone dexamethasone, prednisone, mometasone, fluticasone, beclomethasone, flunisolide, and budesonide; and non-steroidal anti-inflammatory agents, such as fenoprofen, flurbiprofen, ibuprofen, ketoprofen, naproxen, oxaprozin, diclofenac, etodolac, indomethacin, ketorolac, nabumetone, sulindac tolmetin meclofenamate, mefenamic acid, piroxicam, and suprofen.

Various antibiotics can also be employed in accordance with the presently-disclosed subject matter including, but are not limited to: aminoglycosides, such as amikacin, gentamycin, kanamycin, neomycin, netilmicin, paromomycin, streptomycin, or tobramycin; carbapenems, such as ertapenem, imipenem, meropenem; chloramphenicol; fluoroquinolones, such as ciprofloxacin, gatifloxacin, gemifloxacin, grepafloxacin, levofloxacin, lomefloxacin, moxifloxacin, norfloxacin, ofloxacin, sparfloxacin, or trovafloxacin; glycopeptides, such as vancomycin; lincosamides, such as clindamycin; macrolides/ketolides, such as azithromycin, clarithromycin, dirithromycin, erythromycin, or telithromycin; cephalosporins, such as cefadroxil, cefazolin, cephalexin, cephalothin, cephapirin, cephradine, cefaclor, cefamandole, cefonicid, cefotetan, cefoxitin, cefprozil, cefuroxime, loracarbef, cefdinir, cefditoren, cefixime, cefoperazone, cefotaxime, cefpodoxime, ceftazidime, ceftibuten, ceftizoxime, ceftriaxone, or cefepime; monobactams, such as aztreonam; nitroimidazoles, such as metronidazole; oxazolidinones, such as linezolid; penicillins, such as amoxicillin, amoxicillin/clavulanate, ampicillin, ampicillin/sulbactam, bacampicillin, carbenicillin, cloxacillin, dicloxacillin, methicillin, mezlocillin, nafcillin, oxacillin, penicillin G, penicillin V, piperacillin, piperacillin/tazobactam, ticarcillin, or ticarcillin/clavulanate; streptogramins, such as quinupristin/dalfopristin; sulfonamide/folate antagonists, such as sulfamethoxazole/trimethoprim; tetracyclines, such as demeclocycline, doxycycline, minocycline, or tetracycline; azole antifungals, such as clotrimazole, fluconazole, itraconazole, ketoconazole, miconazole, or voriconazole; polyene antifungals, such as amphotericin B or nystatin; echinocandin antifungals, such as caspofungin or micafungin, or other antifungals, such as ciclopirox, flucytosine, griseofulvin, or terbinafine. In some embodiments, silver nanoparticles or silver ions can also be incorporated to the nanofibers of the compositions of the presently-disclosed subject matter for anti-bacterial purposes, and can be incorporated into the nanofibers either by encapsulation or surface deposition.

In some embodiments, various analgesic and/or anesthetic are attached to or otherwise incorporated into the nanofiber scaffolds of the presently-disclosed subject matter. As used herein, the term “analgesic” refers to agents used to relieve pain and, in some embodiments, can be used interchangeably with the term “anti-inflammatory agent” such that the term analgesics can be inclusive of the exemplary anti-inflammatory agents described herein. Exemplary analgesic agents used in accordance with the presently-disclosed subject matter include, but are not limited to: paracetamol and non-steroidal anti-inflammatory agents, COX-2 inhibitors, and opiates, such as morphine, and morphinomimetics.

As used herein, the term “anesthetic” refers to agents used to cause a reversible loss of sensation in subject and can thereby be used to relieve pain. Exemplary anesthetics that can be used in accordance with the presently-disclosed subject matter include, but are not limited to, local anesthetics, such as procaine, amethocaine, cocaine, lidocaine, prilocaine, bupivicaine, levobupivicaine, ropivacaine, mepivacaine, and dibucaine.

Once the desired nanofiber scaffolds have been produced, in some embodiments of the presently-disclosed subject matter two or more nanofiber scaffolds are layered together. By layering multiple nanofiber scaffolds, the superior and unexpected advantages of each nanofiber scaffold can be obtained, and, in some embodiments, produce a synergistic effect. In particular embodiments of the presently-disclosed subject matter, a first nanofiber scaffold including microwells seeded with one or more relevant cells and/or skin tissue is layered on a second nanofiber scaffold having radially-aligned nanofibers. In this particular embodiment, the first nanofiber scaffold can provide the benefit of increasing the repair of damaged skin by providing relevant cells and/or skin tissue whereas the second nanofiber scaffold can provide the benefit of directing and enhancing cell migration from the periphery to the center of the layered nanofiber scaffolds. Layering two or more nanofiber scaffolds can also improve the watertight properties of a nanofiber scaffold. Embodiments of the presently-disclosed subject matter can comprise any number or combination of nanofiber scaffolds to obtained desired characteristics.

Still further provided, in some embodiments of the presently-disclosed subject matter are methods for treating damaged skin. In some embodiments, a method for treating damaged skin in a subject is provided that comprises: providing a composition comprising a first nanofiber scaffold including microwells seeded with one or more relevant cells, a skin tissue, or combinations thereof, and a second nanofiber scaffold layered on the first nanofiber scaffold; and applying an effective amount of the composition to a site of damaged skin in the subject (i.e., a site of skin damage and, optionally, the immediately surrounding area). In some embodiments of the methods for treating damaged skin described herein, the damaged skin is treated by applying an effective amount of the composition directly to the damaged skin. In some embodiments, the nanofiber scaffold is applied to the damaged skin by directly suturing the scaffold to the damaged skin and/or by covering the nanofiber scaffold with an appropriate bandage (e.g., gauze). In some embodiments, the skin treatment can also combine a negative pressure wound therapy (NPWT) technique to further improve the treatment.

The terms “treatment” or “treating,” as used herein include, but are not limited to, inhibiting the progression of damage to a tissue, arresting the development of damage to a tissue, reducing the severity of damage to a tissue, ameliorating or relieving symptoms associated with damage to a tissue, and repairing, regenerating, and/or causing a regression of damaged tissue or one or more of the symptoms associated with a damaged tissue.

The term “subject” is used herein to refer to both human and animal subjects. Thus, veterinary therapeutic uses are provided in accordance with the presently disclosed subject matter. As such, the presently-disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

The term “effective amount” is used herein to refer to an amount of a composition (e.g., a composition comprising a nanofiber scaffold of the presently-disclosed subject matter seeded with one or more relevant cells and/or skin tissue) sufficient to treat a damaged tissue as defined herein (e.g., a reduction in the amount of damaged tissue or an increase in the amount of regeneration of native tissue). Actual amounts of a composition of the presently-disclosed subject matter can be varied so as to apply an amount of the composition that is effective to achieve the desired response for a particular subject and/or application to a particular tissue. The selected amount will depend upon a variety of factors including the activity of the composition, formulation, the route of administration, combination with other drugs or treatments, severity of the condition being treated, and the physical condition and prior medical history of the subject being treated. Determination and adjustment of a therapeutically effective amount, as well as evaluation of when and how to make such adjustments, are known to those of ordinary skill in the art of medicine.

The presently-disclosed subject matter is further illustrated by the following specific but non-limiting examples. Some of the following examples are prophetic, notwithstanding the numerical values, results and/or data referred to and contained in the examples. Additionally, the following examples may include compilations of data that are representative of data gathered at various times during the course of development and experimentation related to the present invention.

### EXAMPLES

### Example 1

### Fabrication and Characterization of Nanofiber Scaffolds having Radially-Aligned Nanofibers

To analyze the fabrication and characterization of nanofiber scaffolds, while controlling the composition, structure, order and alignment of nanofibers, nanofiber scaffolds based on polycaprolactone (PCL) were initially fabricated by electrospinning. PCL was chosen because of its mechanical properties (i.e., low stiffness and high elasticity) and programmable biodegradability. The nanofiber scaffolds were also seeded with cells to characterize the effects of the radially-aligned nanofibers on cell migration.

Briefly, in the fabrication process, PCL (Mw=65 kDa; Sigma-Aldrich, St. Louis, Mo.) was first prepared in a 20% (w/v) mixture with dichloromethane (DCM) and N,N-dimethylformamide (DMF) (Fisher Chemical, Pittsburgh, Pa.) with a volume ratio of 4:1. The PCL fibers were spun at 10-17 kV with a feeding rate of 0.5 mL/h with a 23 gauge needle as the spinneret. A piece of aluminum foil was used as a collector to obtain random nanofiber scaffolds. To produce radially-aligned nanofiber scaffolds, a collector was utilized that consisted of a ring electrode (e.g., metal ring) and a point electrode (e.g., a sharp needle). Electrospun PCL nanofibers were also coated with fibronectin (Millipore, Temecular, Calif.) as follows. The electrospun fiber scaffolds were sterilized by soaking in 70% ethanol overnight and washed three times with phosphate buffered saline (PBS). Then, the scaffolds were immersed in a 0.1% poly-L-lysine (PLL) (Sigma-Aldrich) solution for 1 h at room temperature, followed by washing with PBS (Invitrogen, Carlsbad, Calif.) three times. Subsequently, the samples were immersed in a fibronectin solution (26 μL of 50 μg/mL fibronectin solution diluted with 5 mL of PBS buffer) at 4° C. overnight. Prior to cell seeding, the fibronectin solution was removed and the nanofiber scaffolds were rinsed with PBS. DuraMatrix-Onlay collagen dura substitute membranes were also obtained (Stryker Craniomaxillofacial, Kalamazoo, Mich.).

The PCL nanofiber scaffolds were then sputter-coated with gold before imaging with a scanning electron microscope (Nova 200 NanoLab, FEI, Hillsboro, Oreg.) at an accelerating voltage of 15 kV. Samples prepared for use in cell culture were inserted into a 24-well TCPS culture plate and sterilized by soaking scaffolds in 70% ethanol.

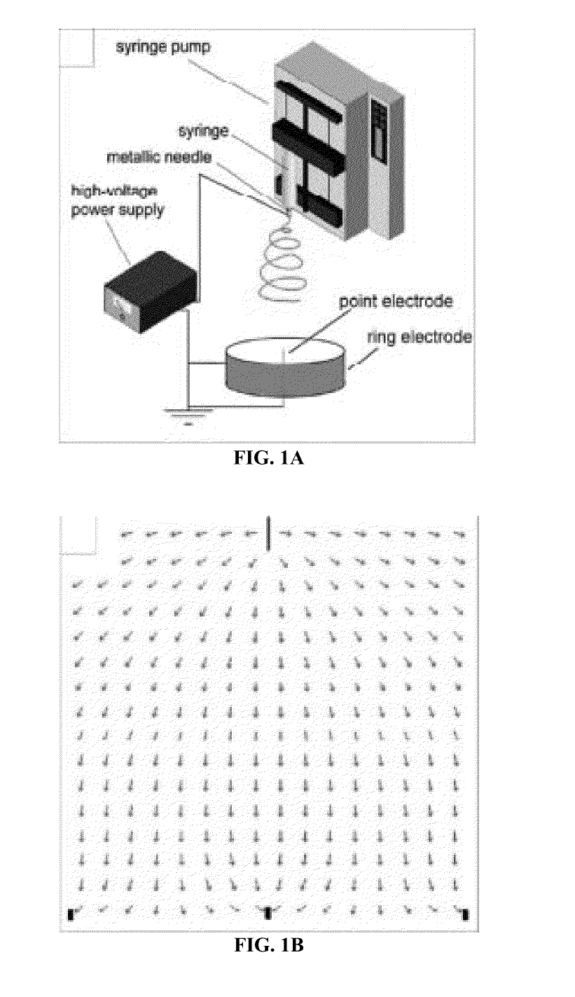

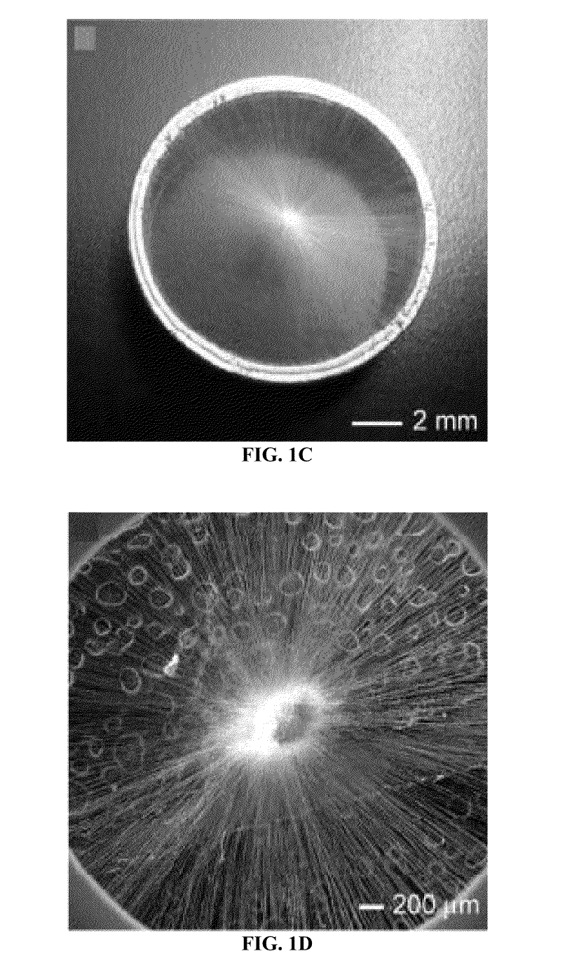

FIG. 1A shows a schematic diagram of the electrospinning setup which consists of a high-voltage generator, a syringe pump, and a collector. FIG. 1B shows a 2D cross-sectional view of the electric field strength vectors between the spinneret and the grounded collector. Unlike conventional systems, the electric field vectors (stream lines) in the vicinity of the collector were split into two fractions, pointing toward both the ring and point electrodes. FIG. 1C shows a photograph of a scaffold consisting of radially-aligned electrospun nanofibers that were directly deposited on the collector. FIG. 1D shows a SEM image taken from the same scaffold, taken with an accelerating voltage of 15 kV, confirming that the nanofibers had been aligned in a radial fashion.

### Example 2

### Characterization of Interface, Cell Adhesion, and Cell Migration in Nanofiber Scaffolds having Radially-Aligned Nanofibers

In order to evaluate the capability of radially-aligned nanofibers to interface with natural dura, promote host cell adhesion to the graft, and enhance host cell migration along the graft, an ex vivo model for the surgical repair of a small dural defect was developed.

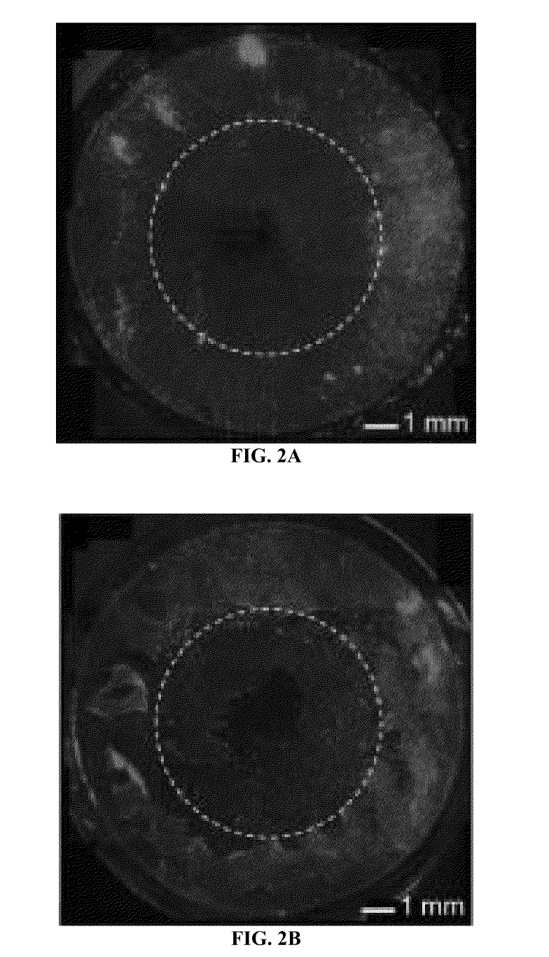

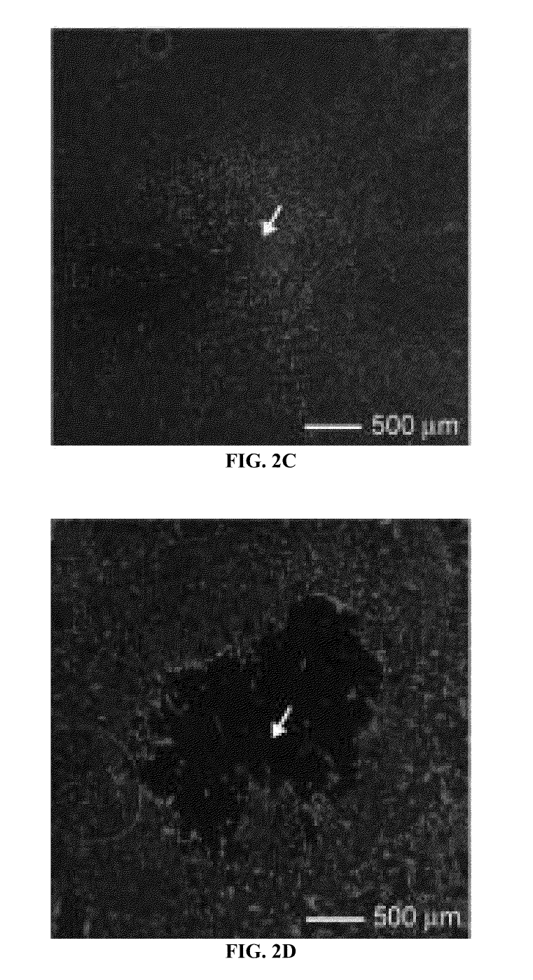

An artificial dural defect was introduced into a piece of dura (1 cm×1 cm) by microsurgically cutting a small circular hole 7 mm in diameter in the center of the specimen. A nanofiber-based scaffold was then overlayed on the artificial defect so as to cover the entire defect and simultaneously contact the dural tissue at the periphery of the specimen. As shown in FIGS. 2A and 2C, dural fibroblasts stained with fluorescein diacetate (FDA) migrated from the surrounding tissue along the radially-aligned nanofibers and further to the center of the circular scaffold after incubation for 4 days. The cells were found to cover the entire surface of the scaffold in 4 days. In contrast, a void was observed after the same period of incubation time for a scaffold made of random fibers (FIGS. 2B and 2D), indicating a faster migration rate for the cells on radially-aligned nanofibers than on their random counterparts. The scaffold made of radially-aligned nanofibers was completely populated with dural cells which had migrated from the borders of the apposed dural tissue. On the contrary, an acellular region at the center of the scaffold was observed after the same incubation time for scaffolds having random nanofibers.

In order to further investigate the effect of fiber alignment and surface coating on cell migration, primary dural fibroblasts were cultured on scaffolds of radially-aligned and random nanofibers without and with fibronectin coating. Briefly, fibroblasts were isolated from sections of dura explanted from 4.5 kg New Zealand rabbits (Myrtle's Rabbitry, Thompsons Station, Tenn.) by first making a 5.0 cm midline incision in the scalp to expose the underlying calvarium. Following periosteal elevation, a 2.5 cm×3.0 cm section of bone was removed from the calvarium to expose the underlying dura. A 2.0 cm×1.5 cm section of dura was then removed through sharp dissection and washed three times with cold PBS. Dural fibroblasts were then isolated by digesting minced dura three times in 4 mL of warm Hank's balanced salt solution (HBSS) containing 0.05% Trypsin and 0.04% EDTA (Sigma-Aldrich, St. Louis, Mo.). Following digestion, collected supernatant was centrifuged and the pellet of dural cells was isolated and resuspended in Dulbecco's modified Eagle's medium (DMEM) supplemented with 10% calf serum and 1% penicillin and streptomycin. Dural cells obtained in this manner were then plated in 75 cm² flasks and expanded (subpassaged no more than five times).

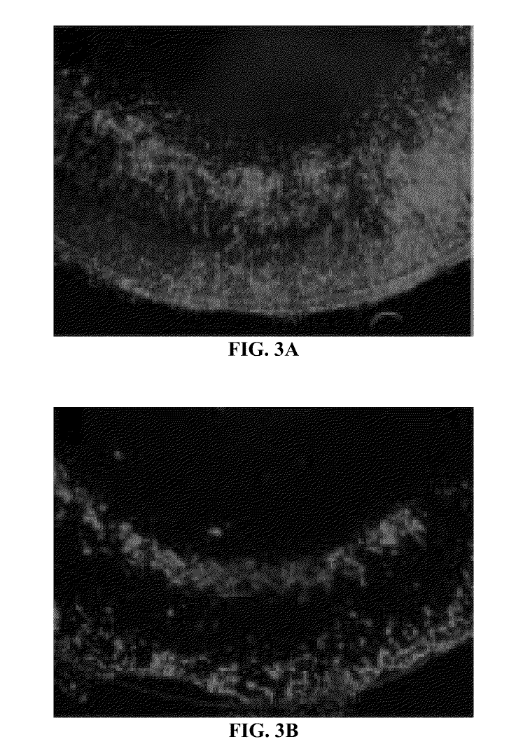

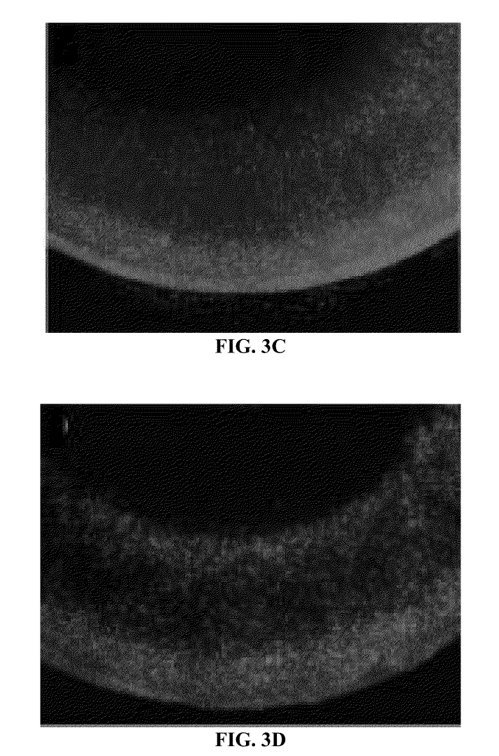

The dural fibroblasts were selectively seeded around the periphery of a circular scaffold of nanofibers, effectively forming a 7 mm simulated dural defect in the center of the sample. A silicone tube was used as a barrier to keep the cells in the ring-shaped area during the first 4 h after cell seeding, and then it was removed. FIGS. 3A to 3D show cell morphology and distribution on scaffolds of radially-aligned and random nanofibers without and with fibronectin coating after incubation for 1 day. As shown in FIG. 3A, many cells attached to the bare scaffold of radially-aligned nanofibers, but fewer cells attached to the bare scaffold of random nanofibers and cell aggregations were noticed (FIG. 3B). The cells were distributed evenly over the entire surface of the fibronectin-coated scaffold of radially-aligned nanofibers, and they exhibited an elongated shape (FIG. 3C). Thus, fibronectin coating could enhance the influence of topographic cues on cell morphology that were rendered by the alignment of fibers. The cells could also adhere well to the fibronectin-coated scaffold consisting of random nanofibers, and cell distribution was more uniform than the uncoated sample (FIG. 3D).

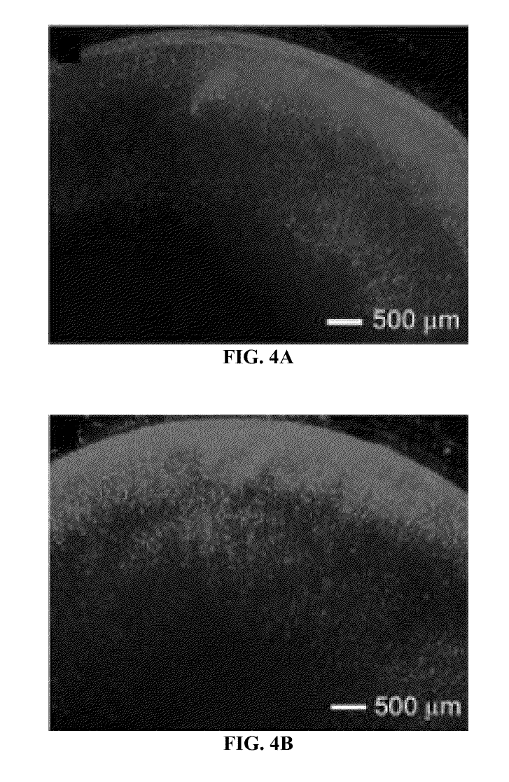

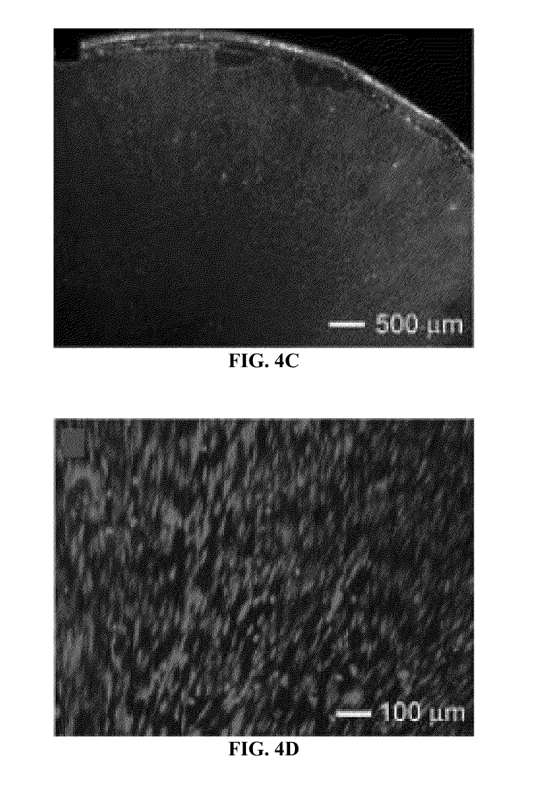

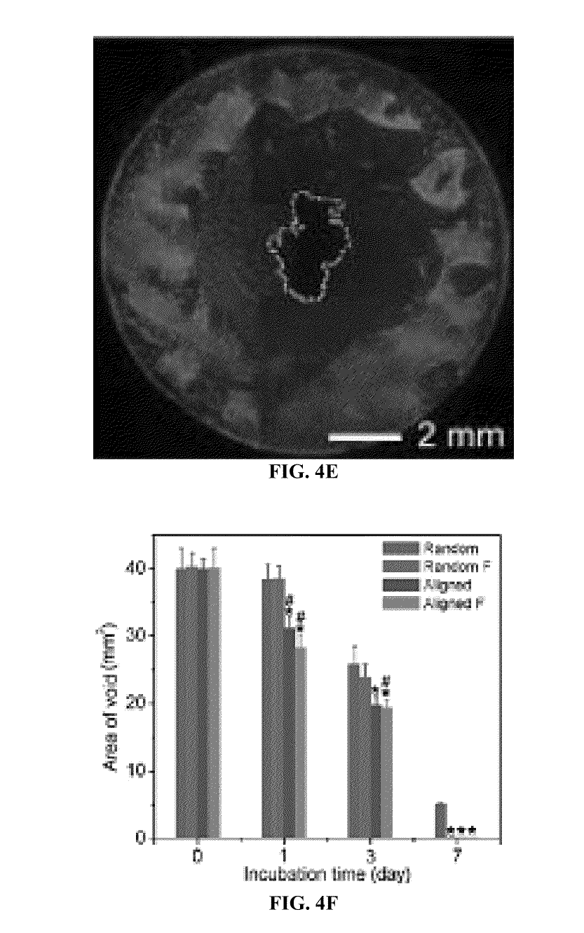

To characterize cell motility on the scaffold, cells were stained with FDA and fluorescence images were taken 1, 3, and 7 days after seeding on different scaffolds, as shown in FIGS. 4A to 4D. The ability for dural fibroblasts to migrate into and repopulate the simulated dural defect was measured at various times throughout the experiment as an estimate of the regenerative capacity of the substitute. FIG. 4E illustrates an example for the calculation of the area of simulated dural defect on the scaffold and the area of void was quantified (FIG. 4F). The area of void decreased with increasing incubation time for all the scaffolds tested due to the inward migration of cells. Radially-aligned fibers significantly enhanced cell migration when compared to random fibers, and cells had the fastest migration rate on the fibronectin-coated scaffold of radially-aligned nanofibers for the first 3 days of incubation. About 5 mm² of bare surface still remained for the bare scaffold of random scaffolds even after incubation for 7 days. In contrast, cells almost entirely covered the area of the simulated defect within the same period of incubation time for the other three types of scaffolds.

### Example 3

### Fabrication and Characterization of Nanofiber Scaffolds having Microwells

Similarly to the process described in Example 1, PCL nanofibers were fabricated by electrospinning. However, the apparatus and technique was modified so that the fabricated nanofiber scaffolds comprised microwells and novel structural cues between the microwells.

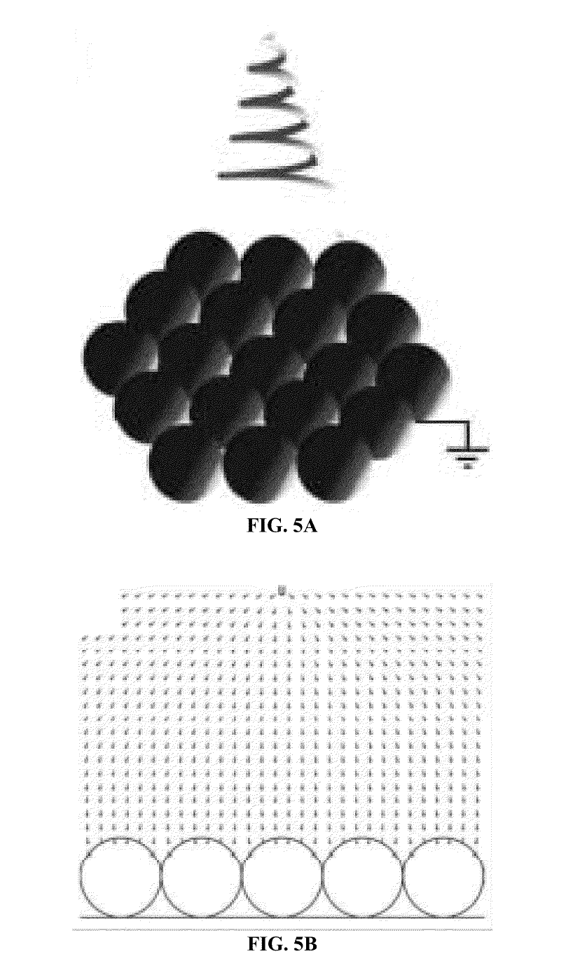

Briefly, and similarly to Example 1, a solution of 20 w/v % PCL (Sigma-Aldrich, St. Louis, Mo.) in a solvent mixture of dichloromethane (DCM) and dimethylformamide (DMF) (Fisher Chemical, Waltham, Mass.) at a volume ratio of 80:20 was prepared. The collector was constructed from stainless steel beads with a diameter of either 1 mm or 2 mm configured into different patterns. FIG. 5A shows a photograph of a nanofiber membrane collected with a close packed array of stainless steel beads 2 mm in diameter. FIG. 5B shows the distribution of electric field between the needle tip and the arrayed metal beads, obtained using the software COMSOL 3.3 (COMSOL Inc., Burlington, Mass.). Note that the electric field vectors above each bead point directly towards the surface of the bead, and the electric field vectors above the gap region between two adjacent beads are split into two main streams, pointing towards each bead. Without being bound by theory or mechanism, this pattern allowed nanofibers deposited directly onto the beads to be randomly oriented while allowing those deposited across the gap between adjacent beads to be uniaxially-aligned.

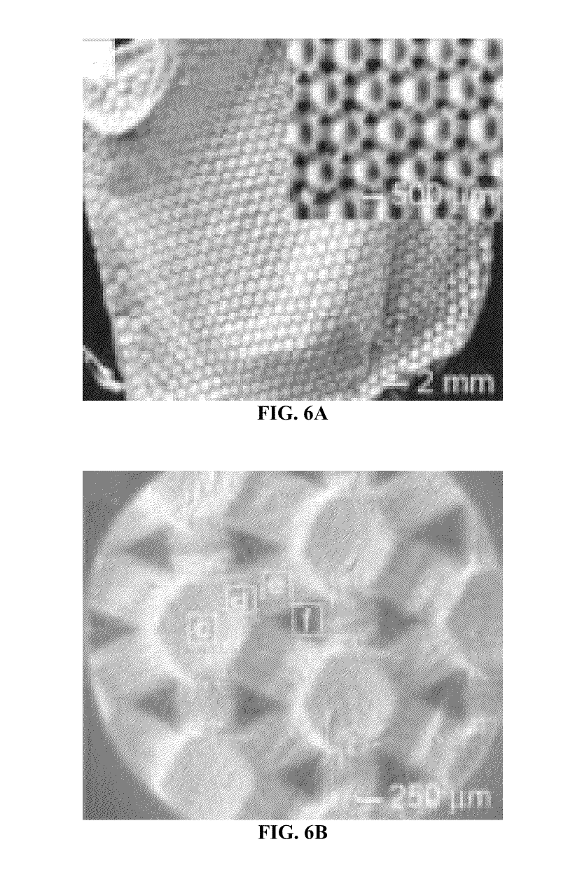

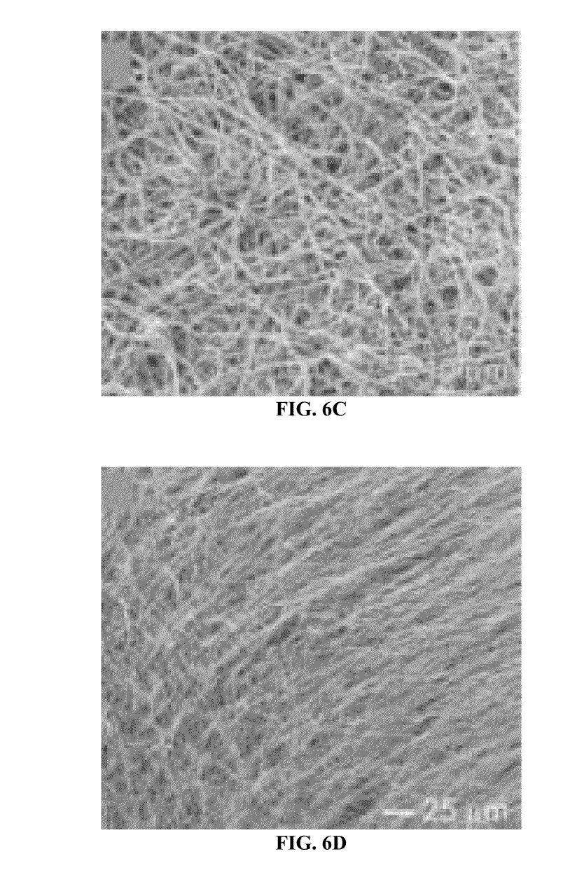

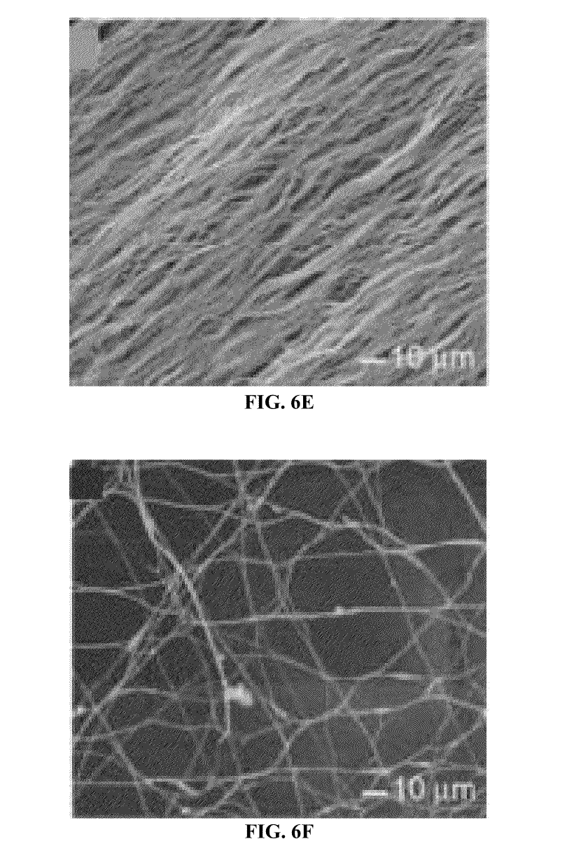

To characterize the nanostructures, the fabricated nanofiber membranes were removed and transferred to culture plates and then fixed by Silastic Type A Medical Adhesive (Dow Corning Co, Midland, Mich.). The PCL nanofibers were sputter-coated with gold and imaged by SEM (200 NanoLab, FEI, Oregon) at an accelerating voltage of 15 kV. FIG. 6A shows a photograph of a nanofiber membrane collected with a close packed array of stainless steel beads 2 mm in diameter. FIGS. 6B to 6E show typical scanning electron microscopy (SEM) images of the same membrane, illustrating a hexagonal array of microwells interconnected through a network of uniaxially-aligned nanofibers. The cross-sectional depth of the wells was observed to be 430±8 μm and 219±6 μm for scaffolds made with 1 mm and 2 mm beads, respectively. It was observed that the nanofibers deposited on the surface of stainless steel beads were randomly oriented whereas those deposited across the gap between two adjacent beads were uniaxially aligned. There was also a short transition zone from random to uniaxial alignment. FIG. 6F shows that the density of fibers was much lower across the void among three neighboring beads than other regions of the membrane and the fibers deposited in the void region were randomly distributed.

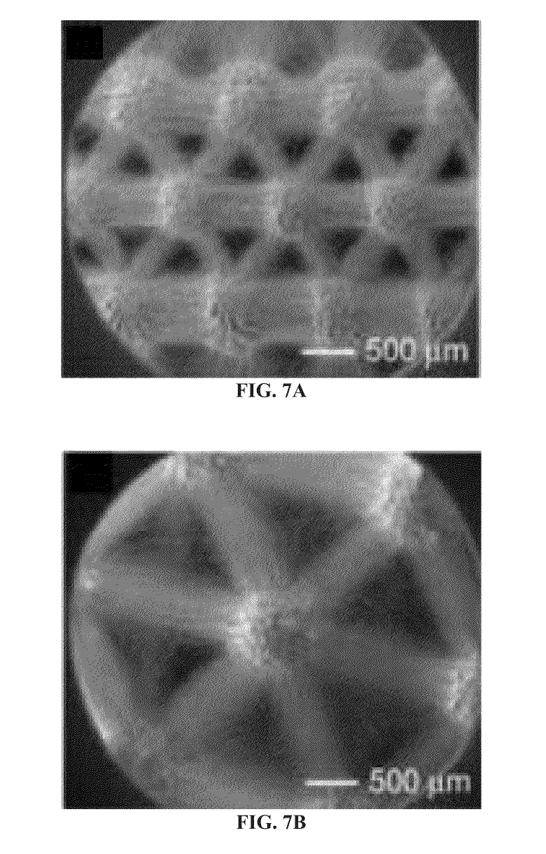

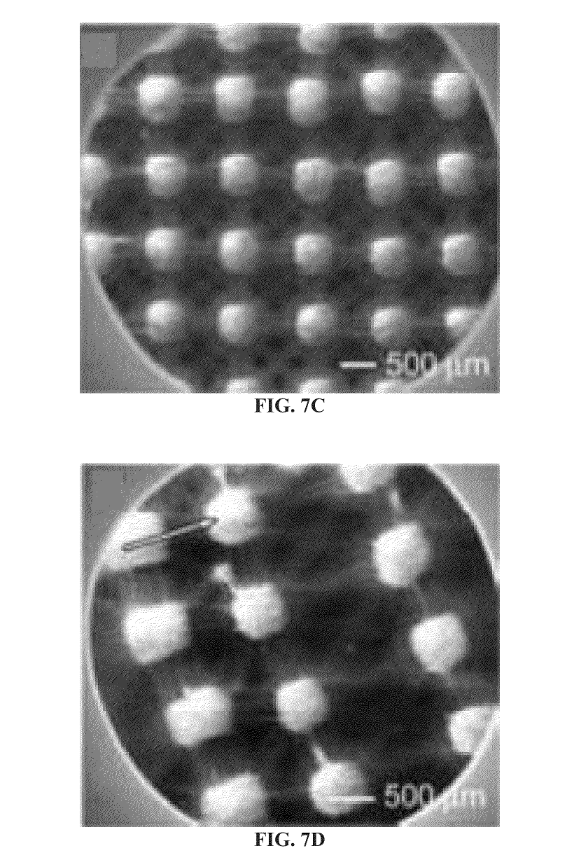

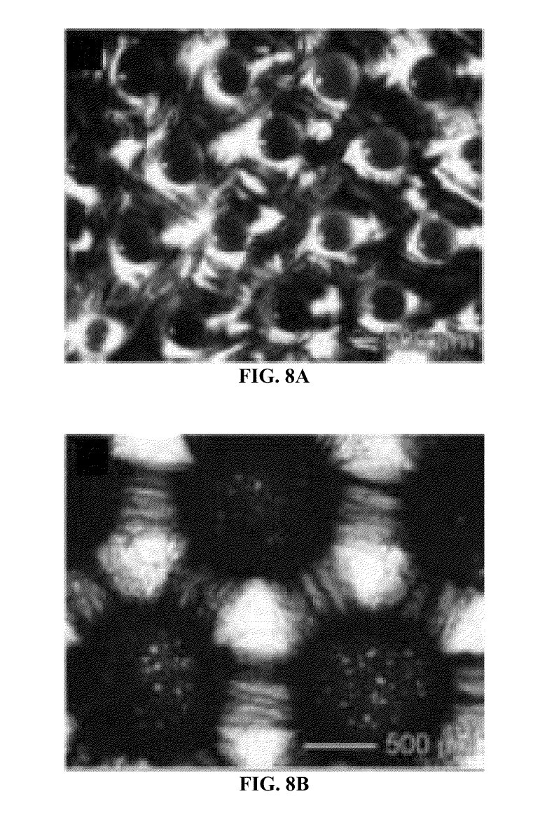

Furthermore, using the above-described technique, additional nanofiber scaffolds were fabricated with different distances between the neighboring wells and well diameters by implementing differently sized beads in various arrangements. For example, FIGS. 7A and 7B show SEM images of nanofiber membranes that were fabricated using hexagonal arrays of stainless steel beads as collectors with different distances between neighboring beads. Other types of arrays besides the hexagonal pattern were also made, including those with square arrays of microwells, as shown in FIG. 7C. FIG. 7D demonstrates the fabrication of a nanofiber membrane with a square array of stainless steel beads as the collectors in which there was a gradual increase in distance between the beads along one direction. For certain applications, the distance between the stainless steel beads is kept below 4 mm and 8 mm for beads with diameters of 1 mm and 2 mm, respectively.

### Example 4

### Characterization of Cells Seeded on Nanofiber Scaffolds having Microwells

The behavior of cells seeded on nanofiber scaffolds having microwells was characterized using the membranes fabricated in accordance with Example 3. A MG63 cell line was obtained from American Type Culture Collection (Manassas, Va.) and was cultured in an alpha—minimum essential medium (α-MEM, Invitrogen), supplemented with 10% fetal bovine serum (FBS, Invitrogen) and 1% antibiotics (containing penicillin and streptomycin, Invitrogen). The medium was changed every other day and the cultures were incubated at 37° C. in a humidified atmosphere containing 5% CO₂.

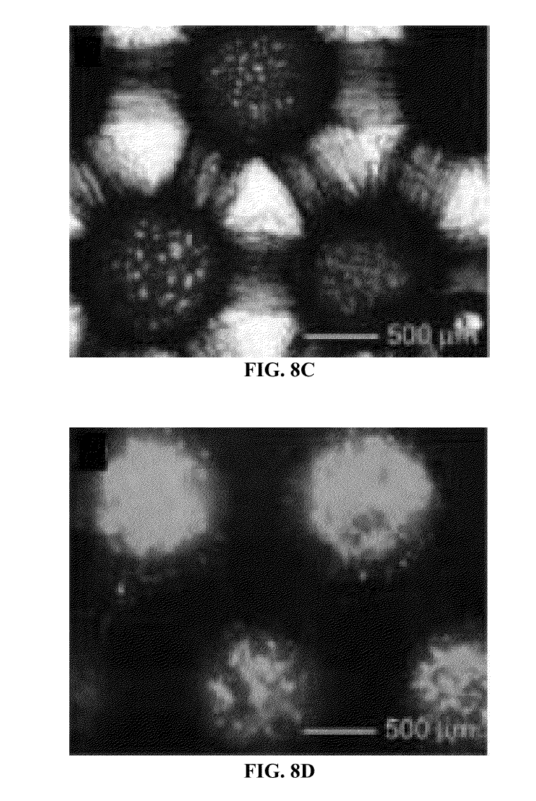

A small droplet containing approximately 50 or approximately 150 cells was loaded directly into each well on the membrane (FIG. 8A). After incubation for 2 h, the membrane was washed with PBS buffer to remove the loosely attached cells. Subsequently, the cell-laden membrane was immersed in the medium and placed into the incubator. After incubation for 24 h or 72 h, the living cells were stained with FDA (Sigma-Aldrich) and imaged with a fluorescence microscope.

FIGS. 8B and 8C show fluorescence microscopy images of a cell microarray fabricated using the nanofiber scaffold, wherein each well contained approximately 45 cells and approximately 150, respectively, and all of the cells were located inside the wells. FIG. 8D shows a fluorescence microscopy image of a third sample, where the initial density of cells was similar to that in FIG. 8C, but the incubation time was increased from one to three days. The cells in the third sample were still physically confined within the wells and the cell microarray was well maintained, although some of the cells likely underwent proliferation during the culture. When cultured for a longer time, the cells would start to migrate from the wells to the regions between the wells, although this could be reduced by forming larger and/or deeper wells.

### Example 5

### Fabrication and Characterization of Further Nanofiber Scaffolds

To analyze further nanofiber scaffolds, an electrospinning setup similar to those described above was used. Briefly, the polymer solution used for electrospinning contained 10 w/v % PCL (Mw: 70,000-90,000, Sigma-Aldrich, St. Louis, Mo.) in a solvent mixture of DCM and DMF (Fisher Chemical, Waltham, Mass.) at a volume ratio of 4:1. The collector was constructed from stainless steel beads with diameters ranging from 0.1 mm to 10 mm, and radially-aligned nanofiber scaffolds were fabricated utilizing a collector consisting of a ring electrode (e.g., metal ring) and a point electrode (e.g., a sharp needle). The fiber membranes were removed and transferred to culture plates and then fixed by Silastic Type A Medical Adhesive (Dow Corning Co, Midland, Mich.). The PCL nanofibers were sputter-coated with gold prior to imaging by scanning electron microscope (200 NanoLab, FEI, Oregon) at an accelerating voltage of 15 kV.

NIH 3T3 cells were purchased from American Type Culture Collection and proliferated in the medium consisting of DMEM plus 10% FBS and 1% gentamicin and streptomycin. Media was changed every other day till confluence. NIH 3T3 cells were seeded on nanofibers and proliferated for 3, 7, 14 days. Prior to cell seeding on nanofiber scaffolds, cells were trypsinized and counted. About 10, 100, or 1000 cells were seeded in each nanofiber microwell. After incubation for 3, 7, or 14 days, live cells were stained using FDA (Sigma, USA). Fluorescent images were then taken using a fluorescence microscope (Zeiss, Thornwood, N.Y., USA).

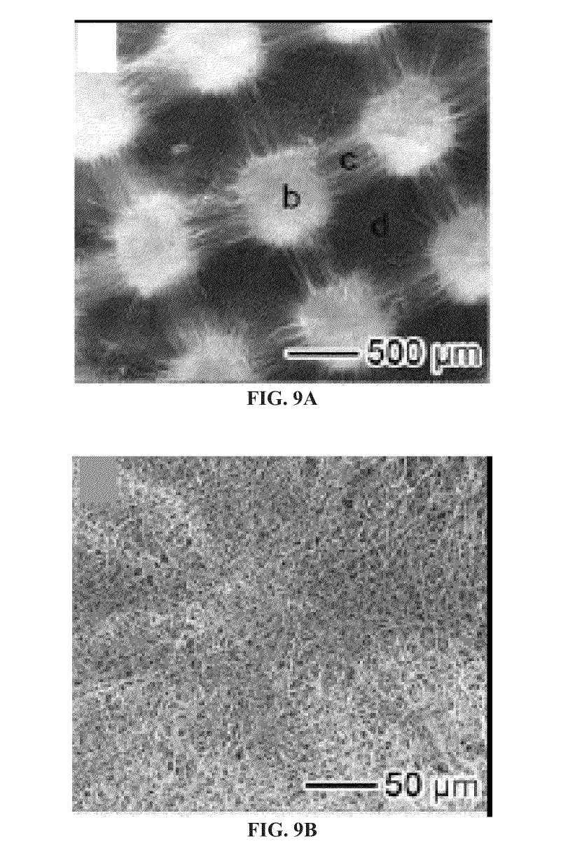

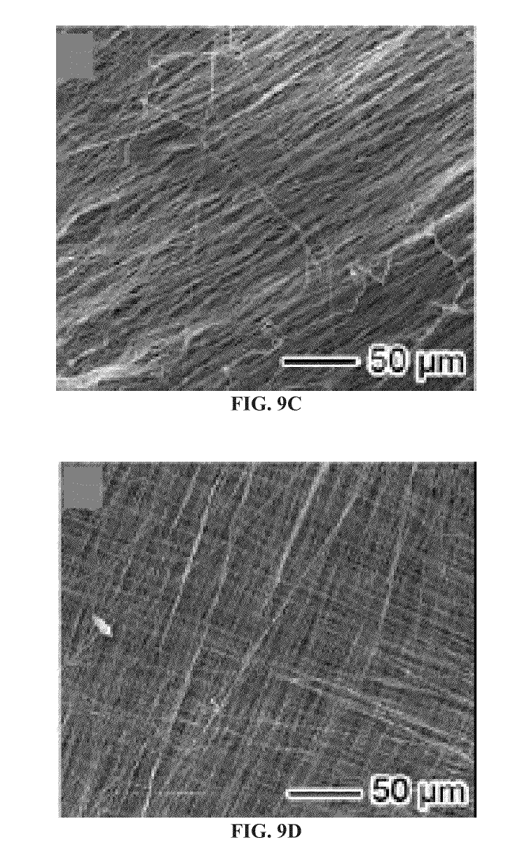

FIGS. 9A to 9D show SEM images of the nanofiber scaffolds, illustrating a complex architecture composed of a square array of microwells interconnected through a network of uniaxially-aligned nanofibers, and randomly-aligned nanofibers deposited on the surface of stainless steel beads.

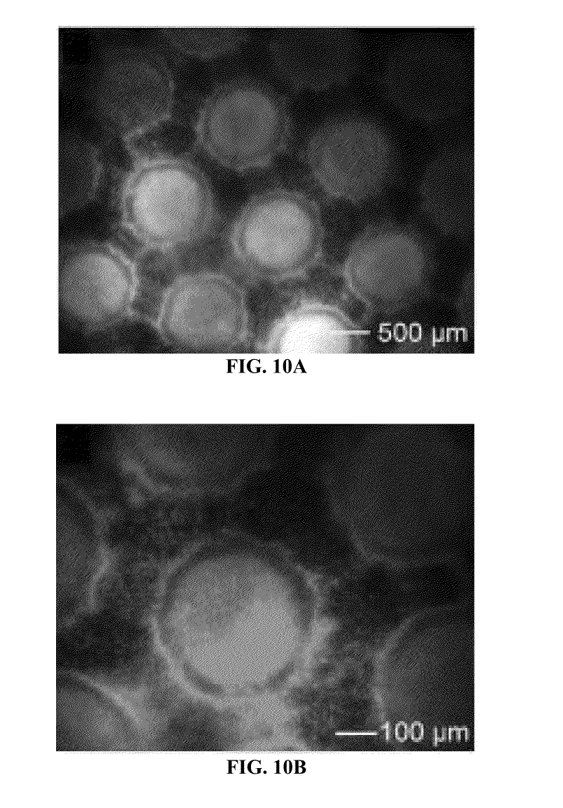

The nanofiber scaffolds having microwells seeded with cells experienced enhanced migration of the seeded cells along the uniaxially-arranged nanofibers located between the nanofibers. FIGS. 10A to 10B illustrate that characteristic with images of live cells stained with FDA that migrated from tissue islands.

Additionally, embryonic day 16 chicks were removed from the eggs and decapitated. The skin tissue was isolated and minced into 1 mm³ pieces. Subsequently, the minced tissues were seeded in to the microwells of nanofiber scaffolds. Thus, such minced skin tissues can be used in lieu of or in combination with seeded cells.

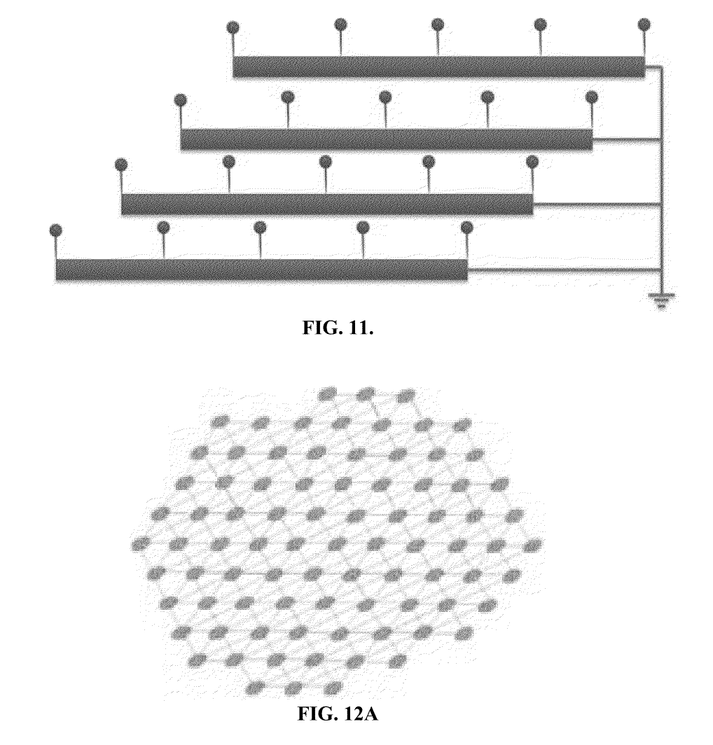

Without wishing to be bound by any particular theory, it was also believed that a large expansion ratio for the nanofiber scaffolds was important for covering wounds over large areas with a small area of skin. Typically, increasing the distance between the skin tissue islands is a simple way to increase expansion ratio. However, during the course and development of the presently-disclosed scaffolds, it was determined that when the distance between metal beads was larger than 3 mm, the aligned nanofibers tended to adhere to the substrate between beads using the assembled metal beads as collector. In this regard, a further design for a collector (arrayed pins capped with metal balls; see FIG. 11) was developed and was surprisingly able to be used to generate nanofiber scaffolds having larger distances (greater than 3 mm) between arrayed microwells.

### Example 6

### Fabrication and Characterization of Multi-Layer Nanofiber Scaffolds

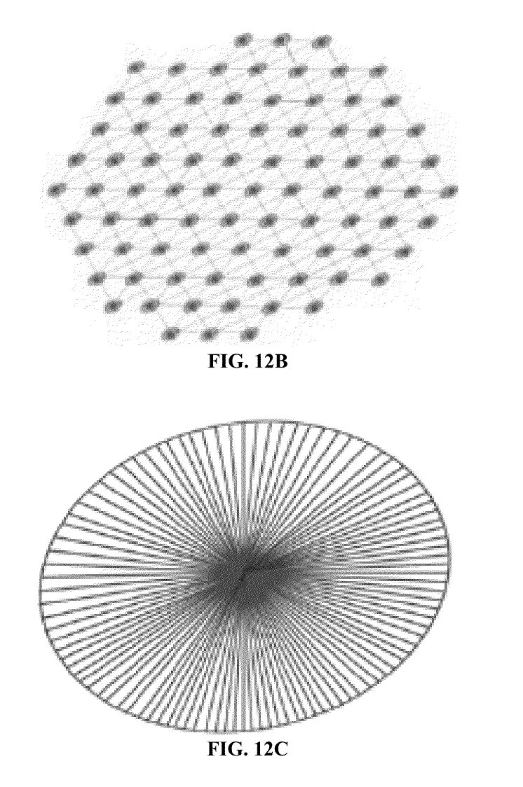

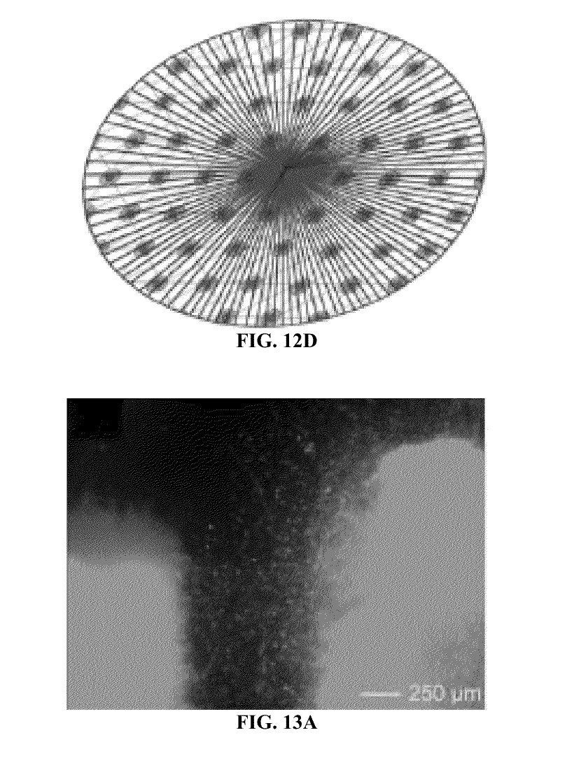

To analyze the characteristics of a multi-layer nanofiber scaffold, scaffolds were fabricated that comprised nanofiber scaffolds having microwells seeded with cells and/or skin tissues as well as nanofiber scaffolds having radially-aligned nanofibers. To better illustrate, FIGS. 12A to 12D are schematic diagrams showing that: a nanofiber scaffold comprising microwells was first fabricated; the microwells were then seeded with minced skin tissue; a nanofiber scaffold having radially-aligned nanofiber scaffolds was then fabricated; and finally the radially-aligned nanofiber scaffold was layered on the nanofiber scaffold comprising the microwells seeded with minced skin tissue.

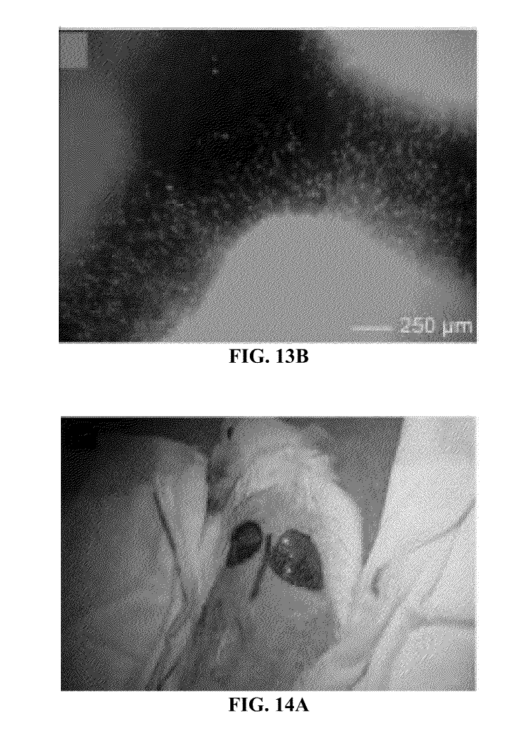

The layering of a radially-aligned nanofiber scaffold on a nanofiber scaffold comprising microwells seeded with minced skin tissues can further enhance and improve the repair of tissue damage and wound healing. For example, FIG. 13A shows the migration of cells that were uniformly seeded on a scaffold having microwells. In comparison, FIG. 13B shows that the combination of scaffolds seeded with embryonic chick skin tissues and radially-aligned nanofiber scaffolds. FIG. 13B illustrates that the cells from the seeded embryonic chick skin tissues were guided by the fiber alignment to the surrounding regions. In other words, the addition of the radially-aligned nanofiber scaffold enhanced the extent to which cells migrated from the microwells of the seeded nanofiber scaffold.

### Example 7

### Treatment of Skin Damage with Dual-Layer Nanofiber Scaffolds

To examine the use of the presently-disclosed nanofiber scaffolds to treat skin damage, the multi-layer nanofiber scaffolds described in Example 6 were examined in an animal cutaneous wound model.

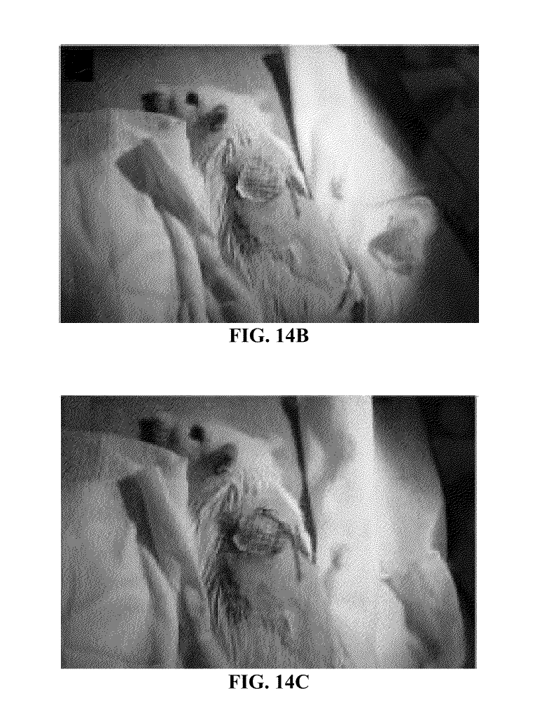

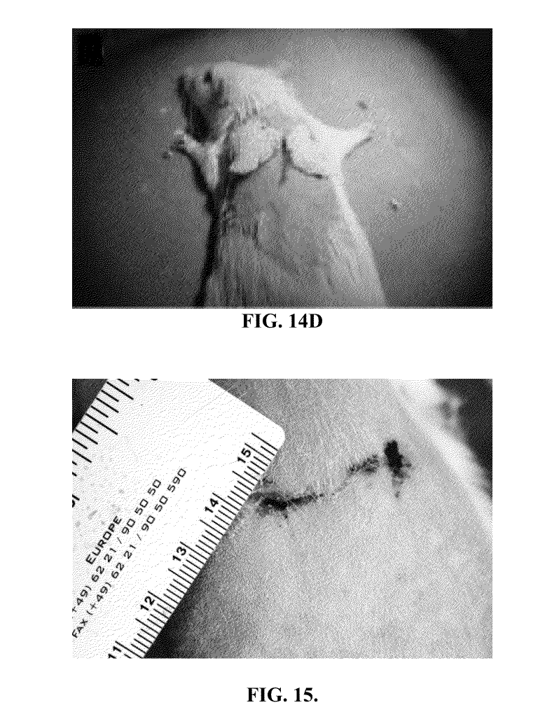

Briefly, male Lewis rats (weight: 250-300 g) were chosen. To generate the rat cutaneous wound model, two full-thickness, 2-cm diameter circular excision wounds were created on each side of the dorsal surface by using a 2-cm diameter circular skin biopsy punch and then by using an Iris scissor (FIG. 14A). Wounds were then covered with a dressing film to protect the wound dryness. As shown in FIGS. 14B and 14C, a sandwich-type nanofiber skin graft was fabricated as follows: cutting of the nanofiber scaffold with arrayed microwells and structural cues about 2 cm in diameter and about 50 μm in thickness; seeding minced skin tissue into the microwells, and placing the radially-aligned fibers on the top. Then, the produced nanofiber skin grafts were placed on the cutaneous wound surface with the radially-aligned nanofiber membrane facing the wound bed, and the grafts were then sutured with 5-0 silk on to the regions of the wound. Lastly, the sutured nanofiber scaffolds were covered with gauze to protect the wound and the nanofiber scaffolds for the remainder of the study.

During the period of post-operative recovery, the rats recovered under an infrared heater and a warming pad until awake and were closely monitored for distress during recovery from anesthesia. The rats were then caged without bedding for the duration of the study. It was observed that the nanofiber scaffold comprising minced skin tissues enhanced and improved the healing of the skin damage relative to a blank nanofiber scaffold (FIG. 15).

### Example 8

### Sandwich-Type Fiber Scaffolds with Square Arrayed Microwells and Nanostructured Cues as Microskin Grafts for Skin Regeneration

Additional experiments were performed to further analyze the multi-layered or ‘sandwich’-type nanofiber scaffolds that included radially-aligned nanofiber scaffolds at the bottom, nanofiber scaffolds with square arrayed microwells and structural cues at the top, and microskin tissue islands seeded in microwells in between. These additional studies using the exemplary nanofiber skin grafts simultaneously presented the following features: i) nanotopographic cues (direct and facilitate cell migration which is not available in the current bioengineered skin products); ii) square arrayed microwells (confine skin islands with a uniform distribution, resulting in better cosmetic appearance after wound healing); iii) large expansion ratio (smaller donor sites needed to cover a large wound area); iv) permanent (not a temporary coverage), v) immediate availability and ease of operation (adhere very well to the wound and thus prevent microskin grafts loss during transplantation which usually occurs in traditional skin grafts on severe burns); and vi) biosafety (FDA approved materials and autologous tissue without immune rejection). In the additional studies, poly(ε-caprolactone) (PCL) was chosen because it could provide the desired biomechanical properties and retain a controllable biodegradability in vivo from several weeks to months by incorporating some enzymes, while the degradation products of PCL are nontoxic and can be eliminated from the body in the form of carbon dioxide and water.

Briefly, to perform the studies, the fabrication of the electrospun nanofiber scaffolds was performed in a typical procedure for electrospinning. PCL (M_{w}=70-90 kDa, Sigma-Aldrich) nanofibers that used a solution of 10% (w/v) PCL in a mixture of dichloromethane (DCM) and N,N-dimethylformamide (DMF) (Fisher Chemical) with a volume ratio of 4:1. The nanofibers were spun at 10-17 kV with a feeding rate of 0.5 mL/h, together with a 23 gauge needle as the spinneret. The scaffolds with square arrayed microwells and structural cues were fabricated using a modified collector which was constructed from stainless steel beads with a diameter of 1.58 mm capped rods which were arranged in a square array and the distances between adjacent beads were 2 mm, 3 mm and 6 mm, respectively. Radially-aligned nanofiber scaffolds were fabricated utilizing a collector consisting of a ring electrode (e.g., metal ring) and a point electrode (e.g., a sharp needle) based on previous work. For in vivo studies, nanofiber scaffold samples were treated with air plasma for 5 minutes by Plasma Cleaner PDC-32G (Harrick Plasma, USA). The nanofiber scaffolds were then sterilized by soaking in 70% ethanol overnight and left to dry in a biosafety cabinet prior to implantation in vivo.

To characterize the nanofiber scaffolds, the morphologies and structures of nanofiber scaffolds were characterized by scanning electron microscopy (SEM) (200 Nanolab, FEI, Oregon). To avoid charging, the PCL nanofiber scaffolds were coated with gold using a putter coater for 40 s in vacuum at a current intensity of 40 mA after the scaffolds had been fixed on a metallic stud with double-sided conductive tape. The accelerating voltage was 15 kV for the imaging process.

To analyze the application of cells to the scaffolds, NIH3T3 cells were cultured in Dulbecco's modified Eagle's medium (DMEM) supplemented with 10% fetal bovine serum (FBS, Invitrogen) and 1% gentamycin/streptomycin (Invitrogen) at 37° C. in an atmosphere of 95% air/5% CO₂. Cell culture medium was replaced every 2 days. Skin cells were also isolated from skin tissues explanted from Lewis Rats (Hilltop Lab Animals, Inc., USA). One full-thickness, 2-cm diameter circular skin excision wounds were created on each side of the dorsal surface using a 2-cm diameter circular skin biopsy punch and then using an Iris scissor. Paniculus carnosus was removed from harvested skin tissues. Part of the harvested skin tissues was fragmented into 1-mm diameter microskin by a 1-mm diameter skin biopsy punch and transplanted to contralateral wound. The skin cells were isolated from the left skin tissues. Specifically, the dermis was first isolated from the epidermis with scalpels and scissors. Then dermis specimens were fragmented into 4 mm² skin pieces. These skin pieces were cultured in a 100-mm² petri dish containing 10 mL of Dulbecco's modified Eagle's medium (DMEM) with 20% fetal bovine serum (FBS) (Sigma-Aldrich, Saint Louis, USA), penicillin (100 UI/mL) and streptomycin (100 μg/mL). The culture dish was maintained in a humidified incubator at 37° C. in an atmosphere of 95% air/5% CO₂ and the culture medium was changed every two days till reaching confluence Skin cells were then plated in 75 cm² flasks and expanded (subpassaged no more than five times).

Prior to cell seeding on fiber scaffolds, cells were trypsinized and counted. Cells were suspended in a desired density in DMEM supplemented with 10% calf serum and 1% penicillin and streptomycin. One μL of cell suspension was carefully seeded into each microwell of scaffolds fixed in a culture dish. Then, the culture dish was placed in the incubator for 2 h followed by adding of culture medium. For sandwich-type scaffolds, the radially-aligned nanofibers were paced on the surface of microwell scaffolds after cell seeding and then fixed by a sterilized polypropylene ring placed on the top. The culture dish was maintained in a humidified incubator at 37° C. in an atmosphere of 95% air/5% CO₂. The cells were cultured for 3, 7, 14, and 21 days, stained with FDA and imaged with fluorescence microscope. Fluorescent images were taken using a QICAM Fast Cooled Mono 12-bit camera (Q Imaging, Burnaby, BC, Canada) attached to an Olympus microscope with OCapture 2.90.1 (Olympus, Tokyo, Japan). The area fraction which was defined by the ratio between the surface area occupied by cells and the surface area of scaffolds was quantified using Image J software (National Institute of Health).

To further analyze the nanofiber scaffolds, a rat skin injury model was used. In that model, all animals received human care in compliance with the Principles of Laboratory Animal Care formulated by the Guide for the Care and Use of Laboratory Animals. Eighteen male Lewis rats (Hilltop Lab Animals, Inc., USA) weighting 250-300 g were used for the study. Anesthesia was performed with an intraperitoneal injection of ketamine (75 mg/kg) and xylazine (25 mg/kg). The hairs on back were removed using an electric shaver. The surgical site was washed with povidone-iodone (Betadine) soap and solution. The area was draped in an aseptic fashion. One 2-cm diameter circular full-thickness skin excisions extending through the panniculus carnosus were created on each side of the dorsal surface using a 2-cm diameter circular skin biopsy punch and an Iris scissor. The harvested skin tissues were fragmented into 1-mm diameter microskin tissues and then seeded to each microwell of scaffolds. Radially-aligned nanofibers were laid on the surface of microskin tissue-seeded microwell membranes to form sandwich-type scaffolds. These scaffolds were applied to the wound with radially-aligned fibers facing wound bed. Wounds were covered by Gauze Pads (Johnson & Johnson Consumer Products Companies, Inc., USA) which were fixed by Rat Jackets (Harvard Apparatus, USA). Self-Adherent Gentle Wrap (CVS Pharmacy, Inc., USA) was additionally applied to prevent the removal of dressings. The wounds covered with petrolatum gauze were taken as control. Post-operative antibiotics (Neosporin) and analgesic (Buprenex 0.03 mg/kg, administered subcutaneously) were given to minimize the chance of infection and discomfort experience.

To perform histology analysis for the wound healing experiments, anesthesia was performed with an intraperitoneal injection of ketamine (75 mg/kg) and xylazine (25 mg/kg) at day 7, 14 and 21 post-operatively. Photographs of the wounds were taken. Subsequently, wounds together with the surrounding skins were collected and fixed in 10% formaldehyde for at least 48 h and embedded in paraffin. Histology analysis was blindly assessed by two independent observers from the wound sections (5 μm thick) stained with hematoxylin and eosin (H&E). Graft ‘take’ was determined by revascularization and re-epithelialization. Wound area was measured by distance between the margins of wound minus total length of epithelialization in the middle by using Olympus DP-SOFT software (Software Imaging Systems, Munster, Germany).

Additional immunostaining of keratinocytes was also performed. Briefly, harvested skin tissues were fixed in 10% buffered formalin for more than 24 h, then embedded in paraffin and cut into 6-μm-thick sections. After deparaffinization and rehydration, antigen unmasking was performed as follows: immerse in 10 mM Sodium Citrate (pH 6.0) and heat using a hot plate at full until almost boiling, maintain the temperature at or just below the boiling point for 10 min, and then allow slides cool down in solution at room temperature for 20 min. After rinsing with water three times, endogenous peroxidase activity was first blocked with 1% hydrogen peroxide at room temperature for 10 min. After rinsing with water three times and PBS once, sections were permeabilized and blocked with PBS (pH 7.4) containing 10% donkey serum and 0.3% Triton X-100 at room temperature for 1 hour, followed by exposure to the keratin, Pan-Abl antibody (1:75, Thermo Scientific, IL, USA) diluted in PBS containing 2% donkey serum at 4 ° C. overnight. After rinsing with PBS (pH 7.4) containing 0.3% Triton X-100 three times, the sections were incubated with a secondary antibody donkey anti-mouse IgG conjugated with cyanine dye 3 (1:400, Jackson ImmunoResearch Laboratories, PA, USA) at room temperature for 1 h. After rinsing with PBS (pH 7.4) containing 0.3% Triton X-100 for three times, the sections were mounted with anti-fade mounting medium (Vector Laboratories Inc., CA, USA). Fluorescent images were taken using a fluorescence microscopy (Zeiss Thornwood, N.Y., USA).

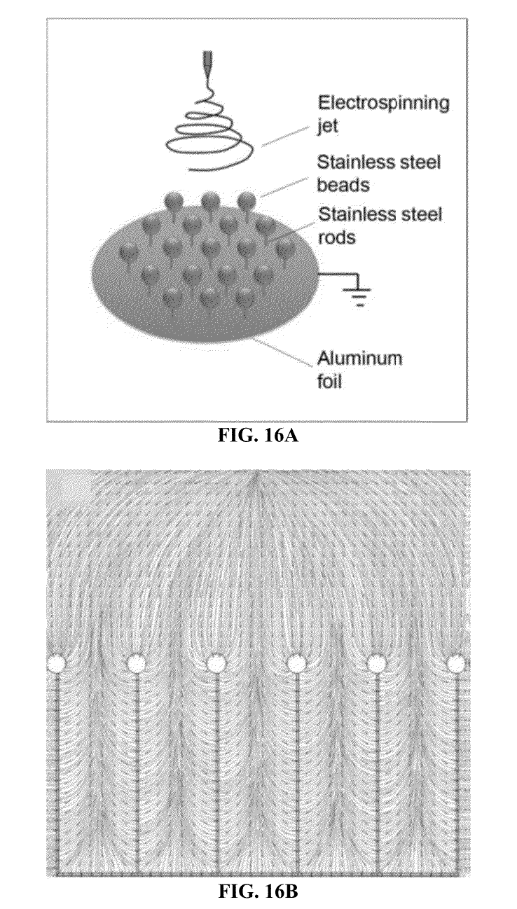

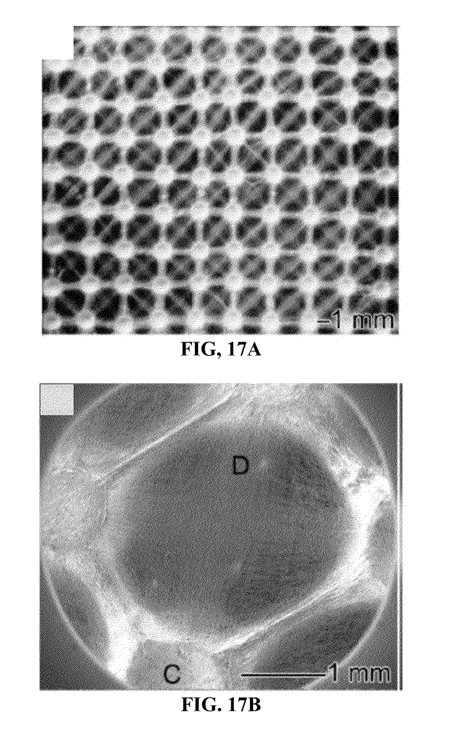

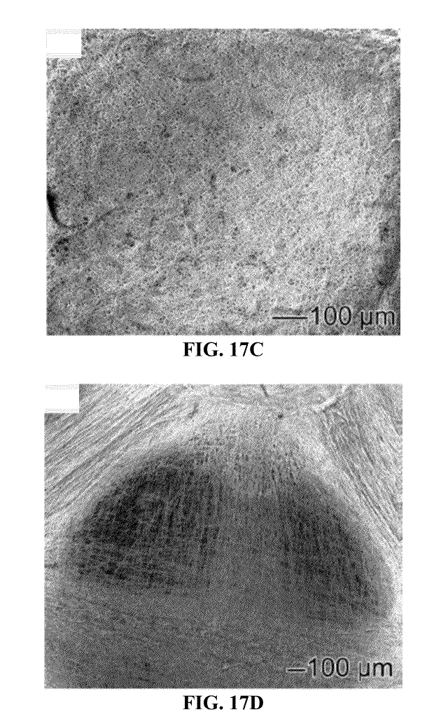

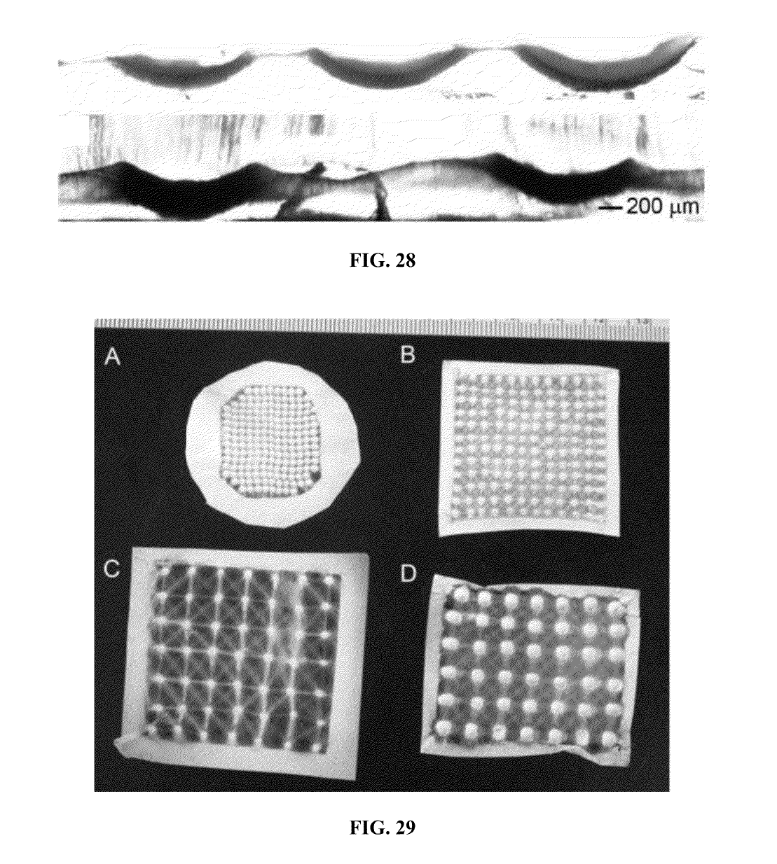

Upon analysis of the results from these studies, it was observed that the nanofiber scaffolds with square arrayed microwells with a distance between adjacent wells larger than 3 mm were successfully assembled using a modified collector (FIG. 16A), which can be assembled from stainless steel bead-capped rods. FIG. 16B shows the distribution of electric field between the needle tip and the arrayed metal bead-capped rods that were attached on aluminum foil, obtained using the software COMSOL 4.3 (COMSOL Inc, Burlington, Mass.). It is noted that the electric field vectors above each bead point directly towards the surface of the bead, similar to a conventional collector. However, the electric field vectors above the gap between two adjacent beads were split into three main streams, pointing towards each bead, rod, and ground respectively. This pattern suggested that the nanofibers deposited directly onto the beads were randomly oriented while those deposited across the gap between adjacent beads were uniaxially-aligned. The deposited uniaxially-aligned nanofibers prevented the nanofibers from depositing towards the rods and ground when the distance between beads was shorter than the distance from bead to ground. This uniquely designed collector enhanced the uniaxial alignment of nanofibers between adjacent beads by avoiding the aligned nanofibers to attach the grounded substrate. FIG. 17A shows a photograph of a typical nanofiber membrane with square arrayed microwells and a triangle area of sparse fibers obtained with a collector composed of an assembly of 1.58-mm diameter stainless steel beads. FIG. 17B shows the scanning electron microscopy (SEM) image of the scaffolds in FIG. 17A, suggesting the nanofiber scaffold had a 3 mm gap between microwells. The image illustrates a complex architecture composed of a square shape array of microwells interconnected through a network of uniaxially aligned nanofibers. Based on our previous study, the depth of the microwells was around 280 μm and 200 μm when the collectors were constructed from 1.58-mm diameter stainless steel beads and the distances between two adjacent beads were 1.58 mm and 3 mm, respectively (FIG. 28). FIGS. 17C-17D show SEM images of the regions indicated in FIG. 17B at higher magnifications. It is clear that the nanofibers deposited on the surface of stainless steel beads were randomly oriented whereas those deposited across the gap between two adjacent beads were uniaxially aligned. In addition, the density of nanofibers was much lower across the void among the align nanofibers than other regions of the scaffold. FIG. 29 shows photographs of the nanofiber membranes with various distances between the neighboring microwells and diameters of microwells, which can be achieved using different assemblies of metal bead-capped rods as collectors.

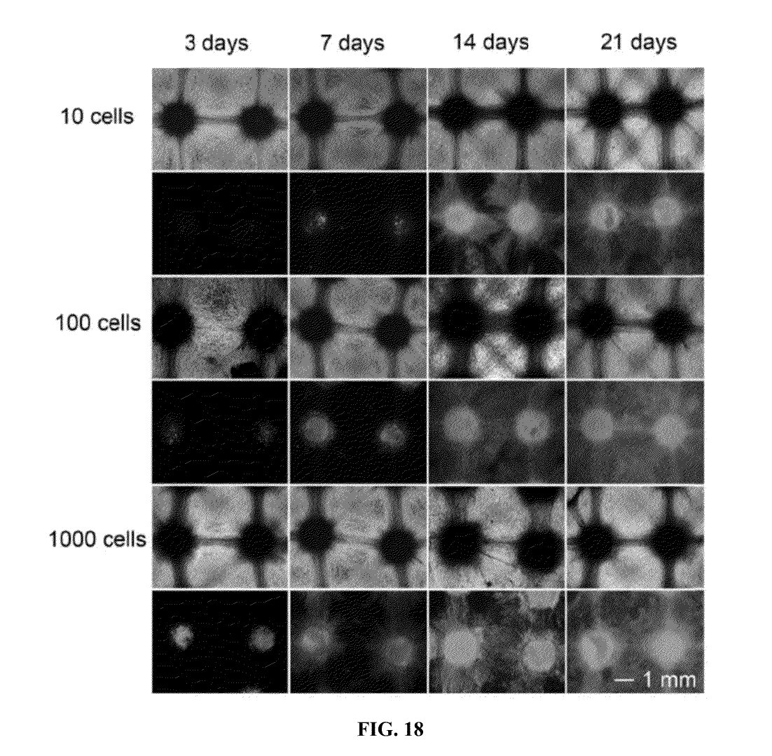

Based on the equation L=x·(1.4\sqrt{Y}−1.2) where L is the distance between the micrograft skin islands, x is the side length of micrograft skin islands and Y is the expansion ratio, the expansion ratio can be tailored by changing the size of skin pieces and distances between skin pieces. Here, different numbers of cells seeded to microwells of nanofiber scaffolds were used to mimic the change of size of skin pieces. In order to evaluate the performance of scaffolds in skin regeneration, we firstly examined the migration and repopulation of NIH 3T3 fibroblasts seeded to individual arrayed microwells of nanofiber scaffolds in different initial cell seeding numbers at different incubation times. The scaffolds with a distance of 3 mm between two adjacent microwells were chosen to demonstrate this proof-of-concept. FIG. 18 shows optical and fluorescence microcopy images of NIH 3T3 fibroblasts plated to arrayed microwells in 10, 100, and 1000 cells per well for 3, 7, 14, and 21 days of incubation respectively. The living cells were stained with fluorescein diacetate (FDA) in green color. Cells were confined within microwells composed of random nanofibers in the first 7 days of culture when seeding 10 cells at the beginning of culture. Only few cells migrated out from microwells in the same time period when initially plating 100 cells to each well. In contrast, many more cells were observed in the region out of the microwells when 1000 cells were seeded to each well. However, no significant difference was observed for all the experimental groups after incubation for 14 days and 21 days. In particular, cells can cover the whole surface of nanofiber scaffolds due to cell migration and repopulation in 21-day period of culture for experimental groups with three different cell seeding numbers.

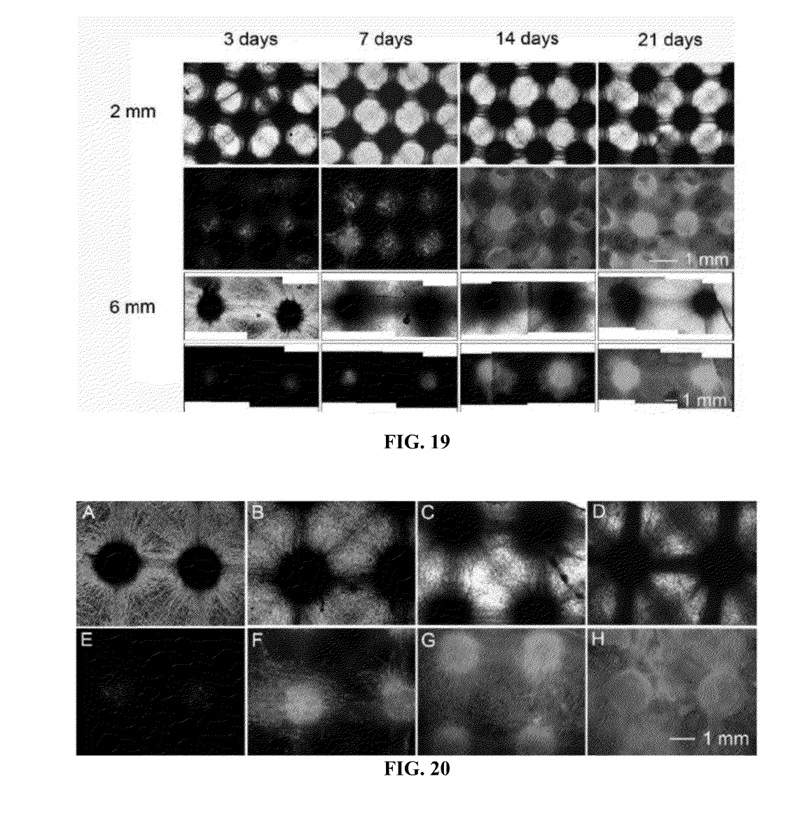

The cell migration and repopulation on nanofiber scaffolds with distances of 2 mm and 6 mm between microwells was further investigated. FIG. 19 shows optical microscopy images and fluorescence microscopy images of 100 NIH 3T3 fibroblasts seeded to each microwell of nanofiber scaffolds with distances between the adjacent wells of 2 mm and 6 mm for 3, 7, 14, 21 days. Similarly, living cells were stained with FDA in green. In the first 14 days of culture, cells migrated to the surrounding regions of wells and repopulated and covered the whole surface of nanofiber membranes when the distance was 2 mm between the two neighboring wells. In contrast, the cells only covered part of the surface of nanofiber membrane when the distance was 6 mm between the neighboring wells. Interestingly, the cells covered the whole surface of both nanofiber membranes after incubation for 21 days.

Recent studies have reported the fabrication of radially-aligned PCL nanofibers which were able to present nanoscale topographic cues to cultured cells, directing and enhancing their migration from the periphery to the center. Here, nanofiber membranes were combined with square arrayed microwells and radially-aligned nanofibers to form sandwich-type scaffolds. Specifically, 100 NIH 3T3 fibroblasts were seeded into each microwell of nanofiber membranes with a 3-mm distance between adjacent microwells and then stacked by radially aligned nanofibers. FIG. 20 shows optical microscopy images and fluorescence microscopy images of NIH 3T3 cells after culture for 3, 7, 14, 21 days on sandwich-type scaffolds. The cells migrated out of microwells composed of random nanofibers towards adjacent microwells in the first 7 days. The cells can cover the whole surface of scaffolds after culture for 14 days and 21 days.

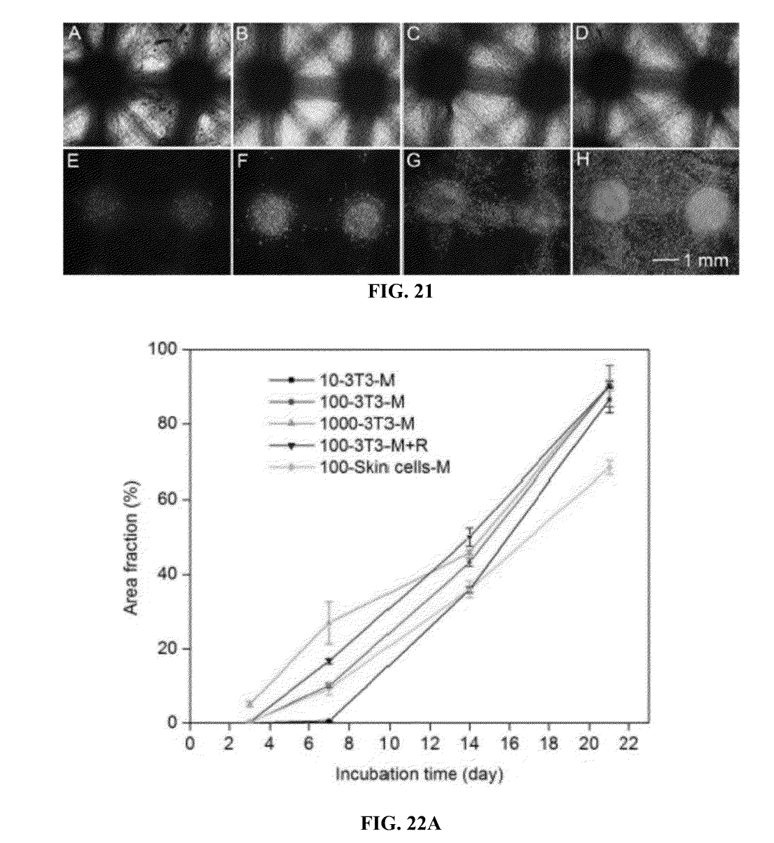

As noted above, the migration and repopulation of primary rat skin cells isolated from rat skins was also examined using nanofiber membranes with square arrayed microwells (FIG. 21). One hundred skin cells were seeded to each microwell of membranes where the distance between adjacent wells was 3 mm. After culture for 7 days, few cells were noticed in the surrounding region of microwells. More cells migrated out from wells following the uniaxially aligned nanofibers and repopulated after culture for 14 days. Significantly, cells can almost cover the whole surface of nanofiber membranes after culture for 21 days.

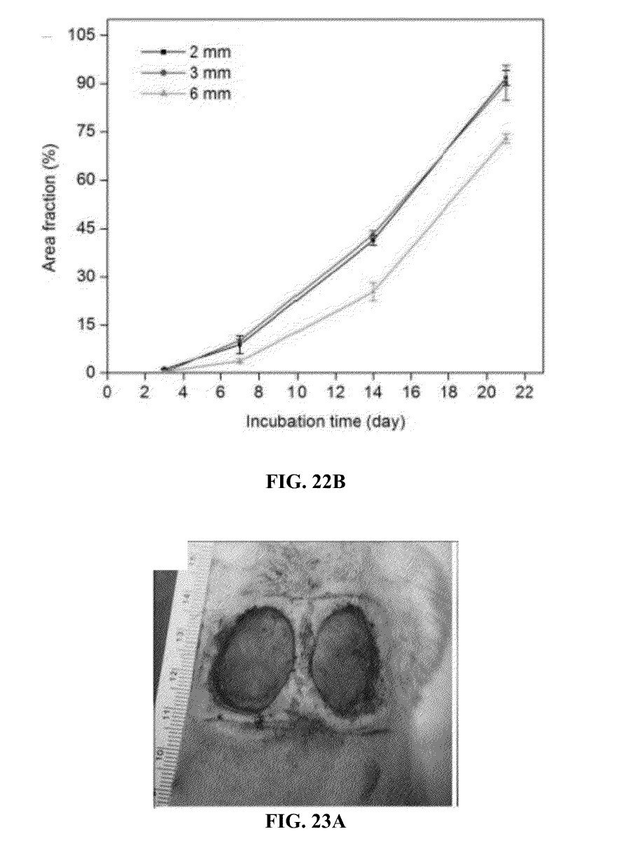

The cell migration and repopulation was further quantified by calculating the ratio of the surface area occupied by cells and the whole surface area of the view using Image J software. The area fraction increased with increasing incubation time for all the experimental groups. For the same period of incubation, higher cell seeding numbers usually resulted in larger area fraction (FIG. 22A). It appeared that the primary rat skin cells migrated and repopulated more slowly than that of NIH 3T3 cells. FIG. 22B showed that smaller distances between the adjacent microwells of nanofiber membranes had higher area fractions.

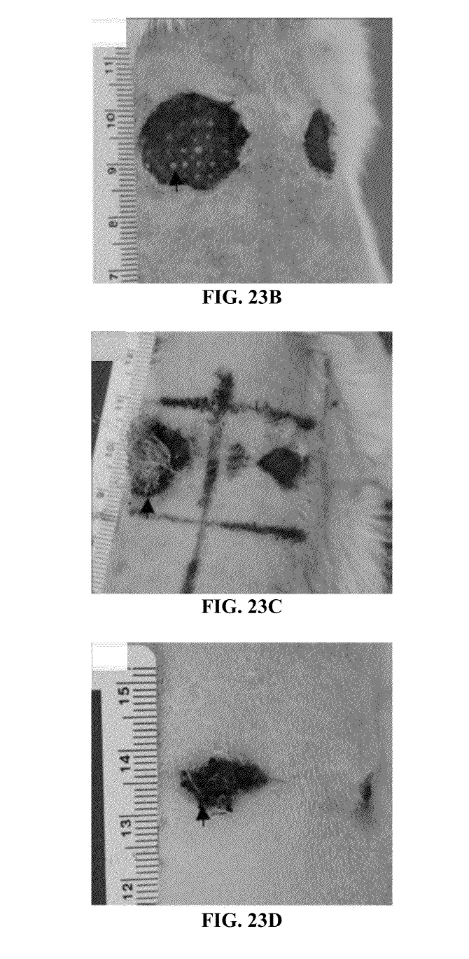

As also noted above, the performance of sandwich-type nanofiber scaffolds were further tested in a rat skin excision model. In order to improve the hydrophilicity and adherence, all the scaffolds were treated with plasma in air for 5 min prior to implantation. With one 20-mm diameter circular full-thickness skin excision wounds created on each side of the dorsal surface of a rat (FIG. 23A), it was observed that sandwich-type nanofiber scaffolds attached tightly to the wound with uniform distribution (microskins indicated by arrow heads) and simultaneously inhibited contraction of wound on day 7 post-surgery (FIG. 23B). FIG. 23C shows sandwich-type nanofiber scaffolds adhered tightly to wound bed, allowing drainage to pass through and adhere to covered gauze dressing on day 14 post-surgery. Besides, the scaffolds significantly inhibited wound contraction in contrast to petrolatum gauze (right side). FIG. 23D shows nanofiber scaffolds adhered well to wound bed even under condition of significant wound contraction on day 21 post-surgery. Similarly, wound contraction was significantly inhibited in contrast to petrolatum gauze.

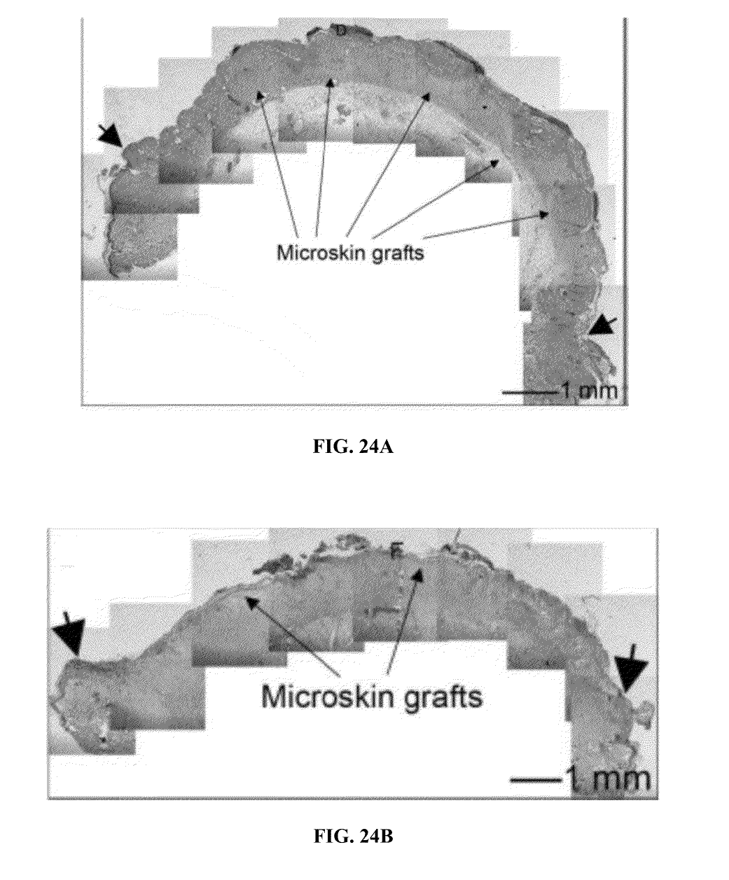

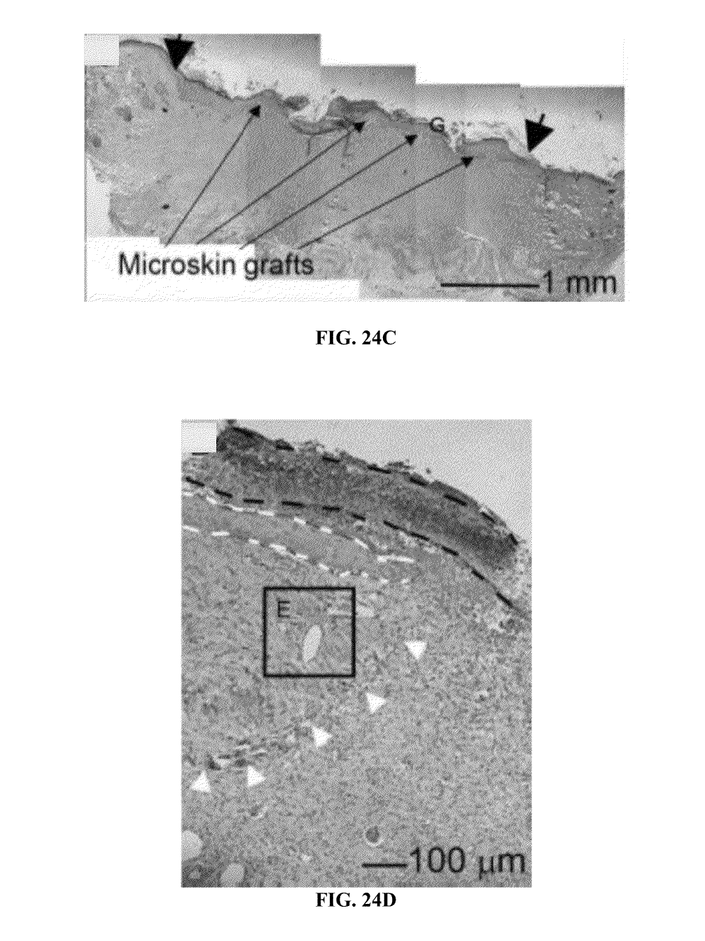

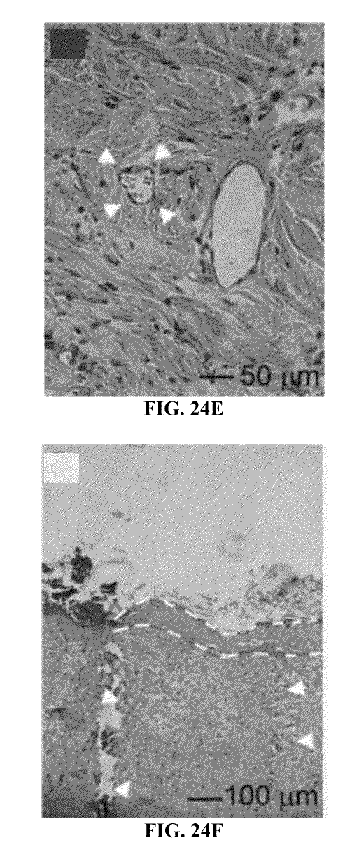

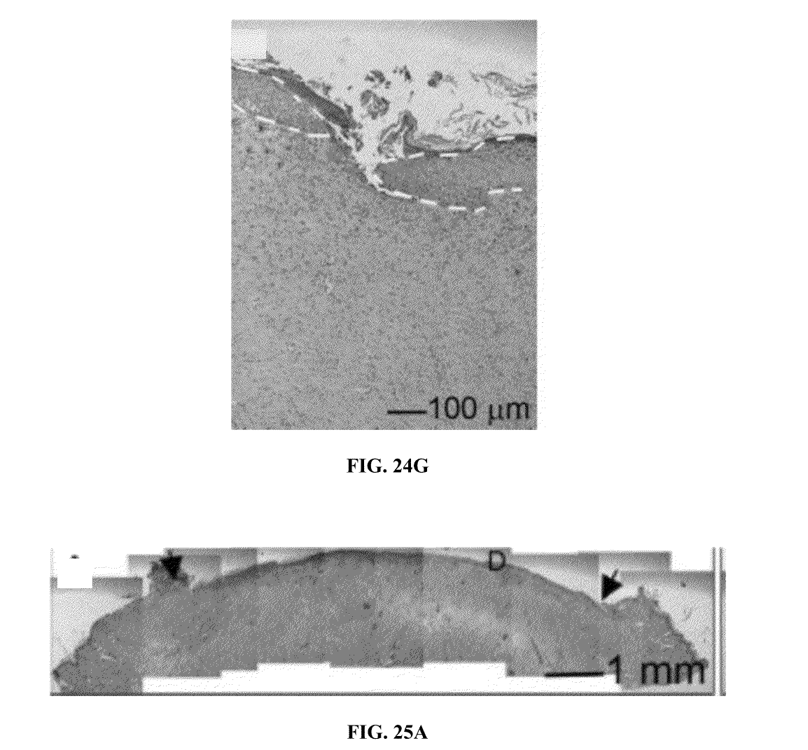

Histological analysis was further performed for the regenerated skin tissues. FIGS. 24A-24G show hematoxylin/eosin (H&E) staining of skin tissue sections, demonstrating that wound healing process was guided by sandwich-type nanofiber scaffolds at day 7, 14 and 21 after surgery. Black arrowheads in FIGS. 24A-24C indicate the boundary between the wound and the surrounding normal skin. FIG. 24A shows all transplanted microskin grafts indicated by small black arrows in sandwich-type nanofiber scaffolds which contained epithelial layer and dermal layer were ‘take’ satisfactorily by wound with a uniform distribution at day 7 post-surgery. It is observed that the microwells composed of random nanofibers were capable of confining the microskin grafts on wound bed. FIG. 24D shows the magnified view of the region D in FIG. 24A, which clearly demonstrating that ‘take’ microskin grafts contained epidermal layer indicated by the area between the two white dash lines and dermal layer indicated by white arrow heads. Epithelial cells derived from cutaneous appendages in microskin graft first developed epidermal cysts or columns and then extended upward to cover the wound surface. A clear boundary was seen between the dermal layer in microskin graft and the granulation tissue of host wound bed. The microwells made of random nanofibers in our scaffolds indicated by black dash lines clearly showed the confinement of microskin grafts on wound bed. Inflammatory cells and red blood cells were also observed between microwells and microskins FIG. 24E shows the magnified view of the region E in FIG. 24D. Small vessels and fresh red blood cells inside indicated by white arrow heads were found in the dermal layer of grafted microskins The re-vascularization could ensure the successful graft ‘take’ by wound. Dermal layer of microskin graft was constituted by collagen bundles and few fibroblasts. FIG. 24B shows re-epithelialization along the wound bed derived from microskin grafts at day 14 after surgery. Epithelial tissues lied in between dry scab and wound bed. FIG. 24F shows the magnified view of the region F in FIG. 24B, suggesting that cells derived from microskin grafts migrated along surface of wound bed towards the neighboring microskin grafts indicated by white dash lines resulting in re-epithelialization. The boundary of dermal layer of microskin graft indicated by white arrow heads started to fade away due to integration with dermal layer of wound bed, suggesting remodeling of microskin grafts was taking place. Epidermis became a stratified epithelium with basement membrane and formed the epithelial tongue indicated by white dash lines in wound repair. FIG. 24C shows that wound were completely closed by re-epithelialization derived from microskin grafts at day 21 after surgery. Dermal layer of the microskin graft indicated by black arrows was completely integrated into surrounding granulation tissue. FIG. 24G an inset in FIG. 24C shows re-epithelialization on wound bed by connecting epithelial cells (indicated by white dash lines) derived from two adjacent skin islands. At the same time, the boundary of dermal layer of the microskin graft disappeared because of their complete integration with surrounding granulation tissues. Epidermis became a mature stratified epithelium with basement membrane indicated by white dash lines and the dry scab fell off from epidermis.

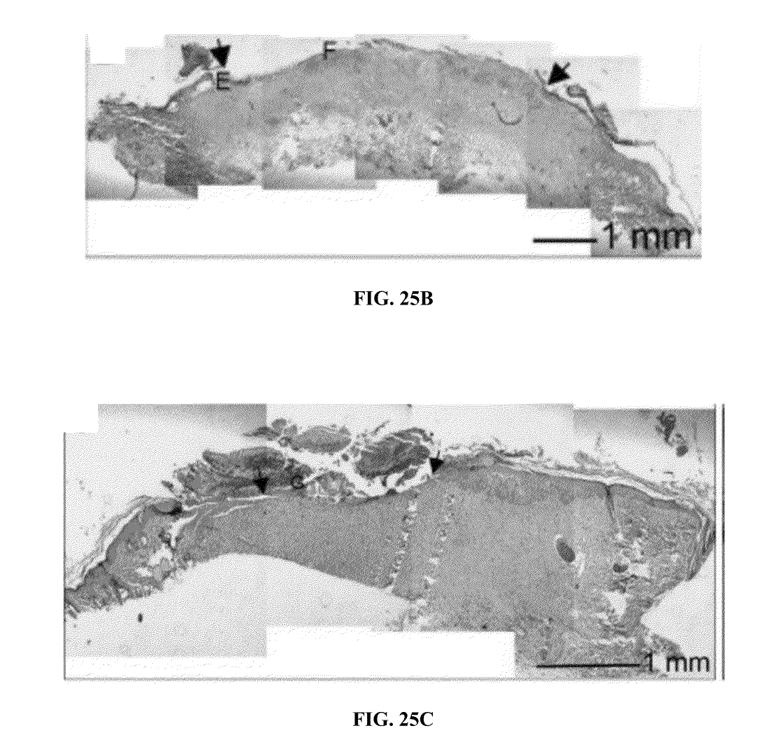

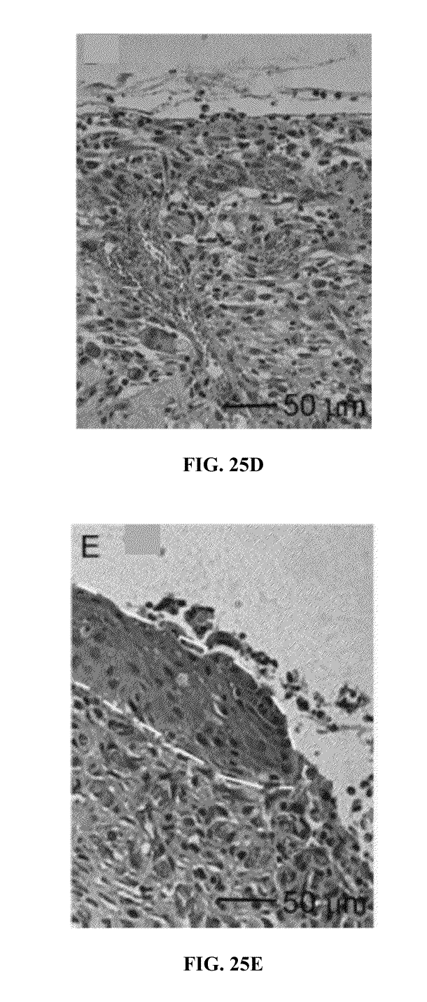

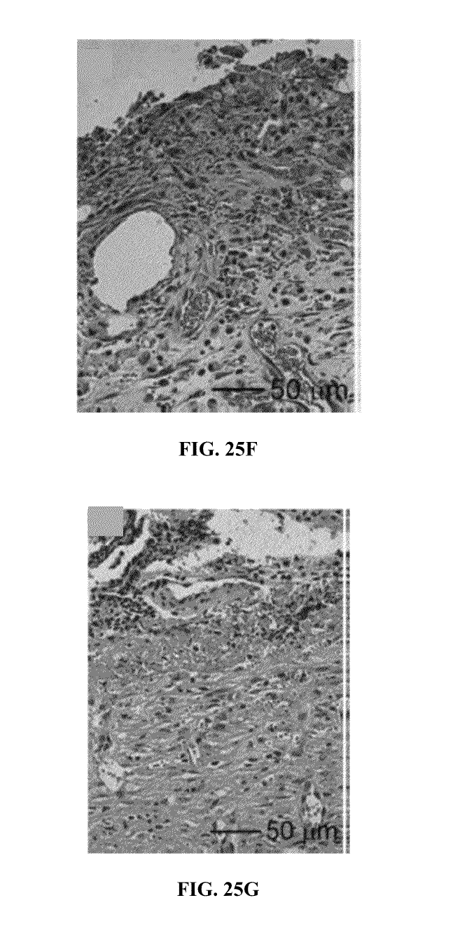

FIGS. 25A-25G show H&E staining of tissue sections, suggesting that the wound healing was resulted from gauze treatment at day 7, 14, and 21 after surgery. Similarly, black arrowheads indicated the boundary between the wound and the surrounding normal skin. FIG. 25A shows epithelial cells were not found on wound at day 7 after surgery. FIG. 25D an inset in FIG. 25A shows a lot of small vessels and fibroblasts grown from wound bed and the wound was repaired by fresh granulation tissue. In contrast, epithelial cell islands were not found on wound bed (FIG. 25B). But epithelial cells migrated from the edge of normal skin toward the center of wound. FIG. 25E an inset in FIG. 25B shows the head of epithelial cell sheet indicated by white dash lines moved to the center of wound on the granulation tissue that contained small vessels and fibroblast, indicating epithelial cells derived from surrounding normal skin migrated along wound bed to repair the wound. FIG. 25F an inset in FIG. 25B suggests that a lot of small vessels and fibroblasts were found on wound bed at day 14 after surgery. FIG. 25C shows a big wound gap still existed at day 21 after surgery and dry scab was detached from wound bed. FIG. 25G (an inset in FIG. 25C) shows the number of small vessels and fibroblasts dramatically decreased and the collagen content significantly increased in granulation tissue.

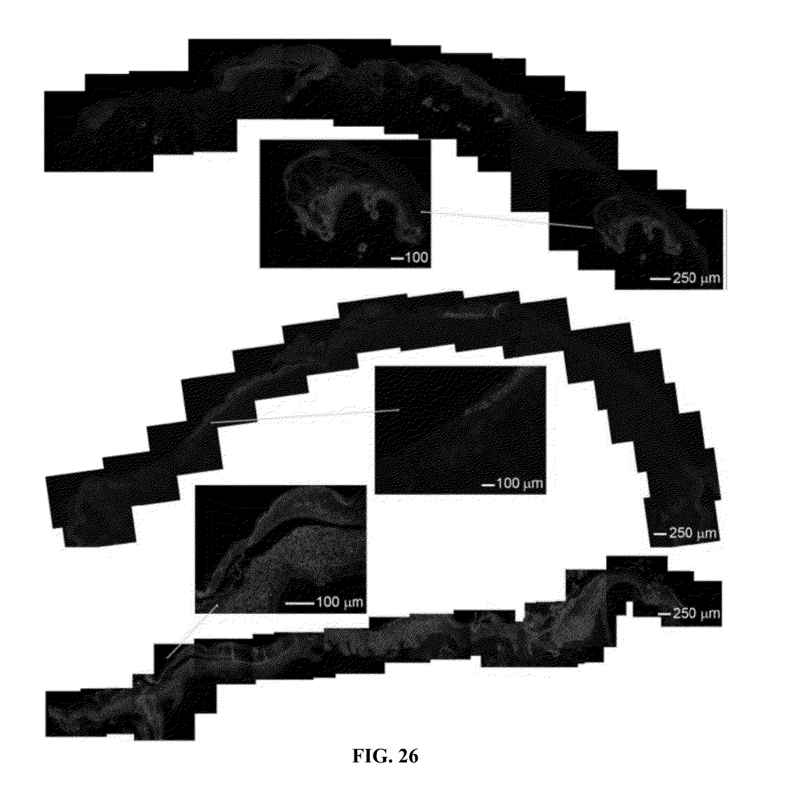

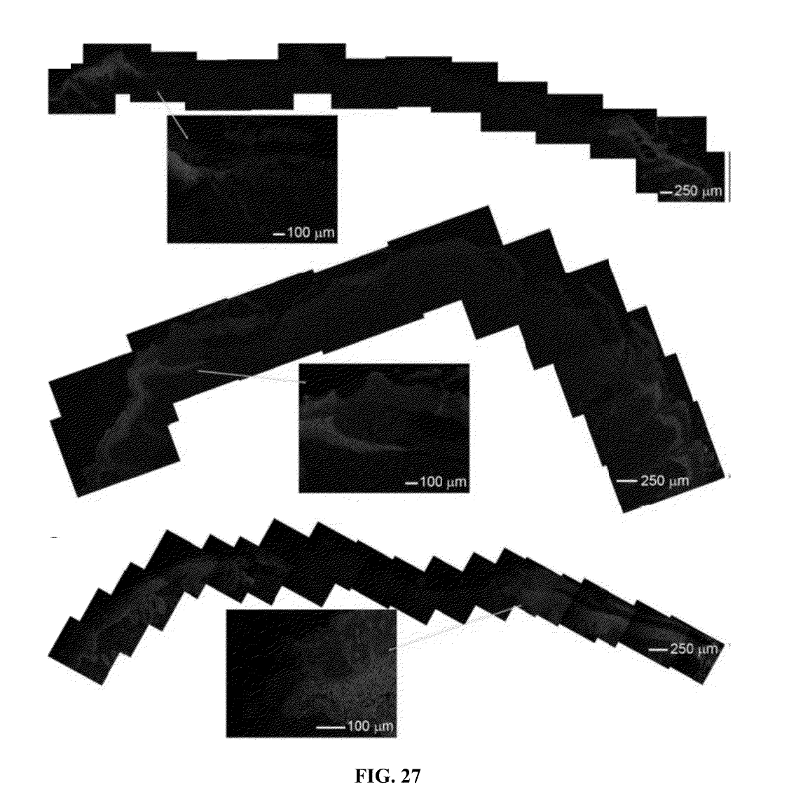

Immunohistochemical staining of epidermal keratinocytes was performed to further reveal the difference of wound healing process when treated with sandwich-type nanofiber skin grafts and petrolatum gauzes, respectively. Keratinocytes were labeled with keratin a keratinocyte marker. FIG. 26 shows immunohistochemistry performed on skin tissue sections after sandwich-type nanofiber skin graft treatment. It is seen that transplanted microskins were ‘take’ after transplanted skins surgery for 7 days. Some of the transplanted microskins can still survive although the epidermal side was not up (inset in upper images in FIG. 26). At day 14 after surgery, keratinocytes migrated from the epidermis of transplanted microskins to the adjacent microskin. The wound closure was mainly attributed to the re-epithelialization of transplanted microskins. At 21 day post-surgery, the wound was completely closed due to the re-epithelialization from microskins and surrounding normal skin (FIG. 26, lower images). FIG. 27 shows immunohistochemistry performed on skin tissue sections after petrolatum gauzes treatment, clearly demonstrating wound closure was attributed to the re-epithelialization from surround normal skin. In contrast, a wound gap was still existed at 21 day post-surgery although the wound size decreased with increasing time after surgery (FIG. 13C).

Efforts have been devoted to the use of electrospun nanofibers in skin regeneration and wound healing. However, most of the studies focused on the creation of skin tissue construct using a tissue engineering approach—a combination of various types of cells, growth factors, antibiotics, and electrospun nanofiber scaffolds. The present study presented an approach combining tissue engineering strategy and microskin graft for healing of skin wounds, in particular, over large areas. Owning to its unique architecture, nanofiber scaffolds developed in the foregoing study presented many advantages over conventional treatments for large burn wounds.

Additionally, the foregoing studies demonstrated that cells seeded to microwells of nanofiber scaffolds can migrate along the long axis of nanofibers, repopulate and cover the whole surface of scaffolds in 3 weeks. And the combination of radially-aligned nanofiber scaffolds could further promote cell migration. The time for cell covering the whole surface of scaffolds was shorter than that (4-5 weeks when the mean epithelialization rate was 90%) for re-epithelialization on severely burned patients treated by MEEK graft. For Meek grafts, microskins are usually placed on the prefolded gauze and expanded in a squared array at a certain distance between two neighboring microskins. The Meek gauzes are now available with expansion ratios of 1:3, 1:4, 1:6 and 1:9. It is known that the expansion ratio is determined by the distance between two adjacent microskins and the size of microskins In the foregoing study, the distance between microwells and the diameter of microwells can be readily tailored by controlling the assembly of metal bead capped pins and the size of metal beads. Therefore, the substrate materials developed in the study provided a flexible choice on the expansion ratio. It was reported that orientation of grafts has marginal influence on skin grafts ‘take’ when the size of skin pieces is smaller than 1 mm³. If the distance between adjacent skin islands is fixed on 6 mm, expansion ratios of 1:25, 1:81, and 1:324 can be acquired when the size of skin grafts is 1×1 mm, 0.5×0.5 mm, and 0.25×0.25 mm. Therefore, a large area of burn wound repaired by a limited donor skin could be realized.

Further, microwells presented from nanofiber scaffolds were capable of confining microskins in a square arrayed pattern and adhered very well to the wound bed without applying pressure, which could ensure the uniform epithelialization. And microwells seemed to be able to enrich nutrition, providing a microniche or 3D cellular microenvironment for blood vessel formation and revascularization and subsequently resulting in an increase of the microskin ‘take’ rate. Without wishing to be bound by any particular theory, this was believed to be important because only serous fluid secreted from wound supports the traditional STSG grafts without blood supply in early 3-4 days and the blood flows through the anastomoses into the vessels in grafts on day 3-4 and proceeds slowly until day 5-6. Additionally, the diameter and depth of microwells in the scaffolds can be tailored by varying the diameter of metal beads and distance between adjacent beads. Such microwell structure could be used to mimic the native three-dimensional (3D) cellular microenvironment at the dermal-epidermal junction (DEJ) as DEJ conforms to a series of 3D rete ridges and papillary projections of the dermis, ranging 50-400 μm in width and 50-200 μm in depth.

Wound beds usually produced large amount of exudates and the graft failure is mainly caused by the accumulation of blood or fluid under skin graft at early stage. The triangle area in the nanofiber scaffolds developed in the foregoing study were composed of sparse fibers, which can effectively facilitate the removal of exudates. It was also observed that the nanofiber scaffolds after plasma treatment can perfectly adhere to the wound bed during wound healing process which could be due to their hydrophilicity and dispersive adhesion. Again without wishing to be bound by any particular theory, it was also though that inflammatory cells or repairing cells could infiltrate into random nanofibers following fluid exudates secreted from wound and these cells can produce the human cathelicidin anti-microbial protein, which not only has broad anti-microbial activity but also induce re-epithelialization of wound.

Currently, re-epithelialization through STSG or CEA graft is the most popular method for wound healing in clinical practice. Unfortunately, CEA graft take is not satisfying in repairing burn wounds. Clinical studies showed chronic granulating wounds had a 15% ‘take’ and freshly excised or early granulating wounds had a 28-47% ‘take’. Histology results in the foregoing study suggested a much higher microskin graft ‘take’ rate close to 100%. Wound healing process can be divided into several phases: contraction, inflammation, granulation tissue remodeling and re-epithelialization. It is impossible for serve burn to heal through pure wound contraction or re-epithelialization from surrounding normal skin. In the foregoing study, wound healing was partly attributed to the wound contraction and epithelialization from surrounding normal skin though nanofiber scaffolds appeared to inhibit wound contraction to some extent. The boundary between the dermal layer of transplanted microskins and surrounding granule tissues was evident at day 7, started to fade away at day 14,m and completely disappeared at day 21 post-surgery, suggesting the occurrence of tissue remodeling. In addition, wound re-epithelialization was also noted from the microskin grafts carried by nanofiber scaffolds. Most interestingly, it was found that the re-epithelialization was taking place inside the granule tissue instead of on the surface of wound bed. After the maturation of epidermis, the granule tissues on the surface of wound bed became dry scabs and fell off. This phenomenon was different from the wound healing mechanism reported in previous studies.

The foregoing studies demonstrated the fabrication of sandwich-type nanofiber scaffolds as microskin grafts with square arrayed microwells and nanotopographic cues and explored their potential applications in skin injury repair. It was also demonstrated that NIH 3T3 fibroblasts with initial seeding densities of 10, 100 and 1000 cells per microwell can migrate, repopulate and cover the whole surface of scaffolds within 21 days in vitro when the distances between two adjacent microwells were 3 and 6 mm, respectively. In addition, primary rat skin cells showed the similar behavior as NIH 3T3 fibroblasts. It was further demonstrated that the sandwich-type nanofiber scaffolds were capable of presenting a uniform distribution of microskin grafts, enhancing the ‘take’ rate of microskin grafts and accelerating re-epithelialization on wounds in a rat skin excision injury model. Taken together, these results indicated sandwich-type nanofiber scaffolds could offer a better solution in skin regeneration on severe burns and provide a suitable carrier for STSG graft in skin regeneration for acute skin defects or chronic wounds

### Example 9

### Analysis of Cell Migration on Nanofiber Assemblies

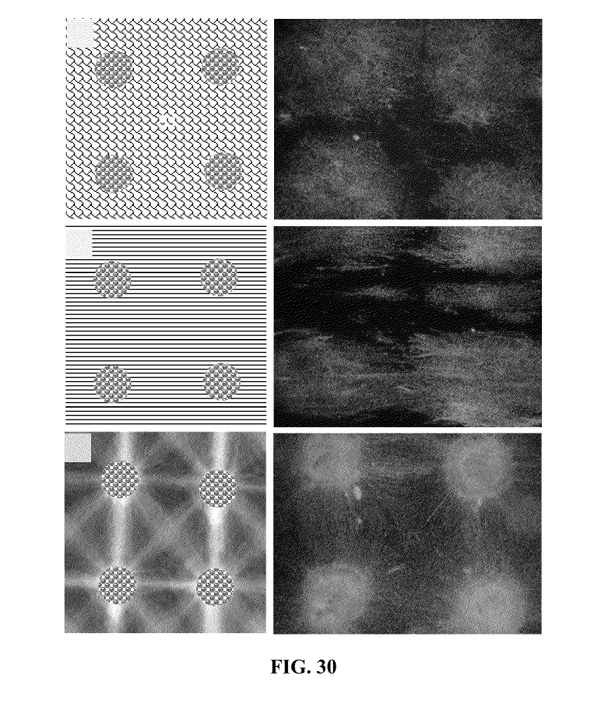

To further examine the capabilities of the presently-disclosed scaffolds, NIH 3T3 fibroblasts were also cultured on different PCL nanofiber assemblies including random nanofibers (FIG. 30, top panels), uniaxially-aligned nanofibers (FIG. 30, middle panels), and exemplary multi-layer nanofiber scaffolds of the presently-disclosed subject matter with arrayed microwells and structural cues (FIG. 30, bottom panels) for 14 days. Briefly, in these experiments, 200 cells were seeded to each microwell at the beginning of incubation, and the distance between the two adjacent microwells/cell spots was 3 mm, and the diameter of the microwells were 1 mm. The microwells (dents) on the random and aligned fiber mats were generated by gently pushing metal beads into the mats. Following a culturing period the living cells were stained with fluorescein diacetate (FDA) an visualized.

Upon anlysis of the results, it was found that the data indicated that cells can cover a whole given area more quickly and distribute more evenly on the nanofiber scaffolds with square-arrayed microwells and structural cues compared to the random and uniaxially-aligned fiber samples. The data also showed that cells can migrate from cell clusters or skin tissue islands seeded iniatially to the microwells to the surrounding areas. Owing to the migration and repopulation, cells can cover the whole surface in as little as 2 weeks.

Throughout this document, various publications, patents, and patent applications are mentioned. All such references are incorporated herein by reference, including the references set forth in the following list:

### REFERENCES

1. Atiyeth B S, Gunn S W A, Hayek S N. Military and civilian burn injuries during armed conflicts, Annals of Burns and Fire Disasters 20: 203-215, 2007.
2. American Burn Association, Burn Incidence Fact Sheet.
3. Ehrenreich W H, Ruszczak Z. Update on tissue-engineered biological dressing, Tissue Eng. 12: 1-18, 2006.
4. Martin P. Wound healing-Aiming for Perfect Skin Regeneration, Science 276: 75-81, 1997.
5. Ehrenfried A. Reverdin and other methods of skin grafting. Boston Med. Surg. J. 26: 911-917, 1909.
6. Beele H, Naeyaert J M, Goeteyn M, et al. Repeated culture epidermal allografts in the treatment of chronic leg ulcers of various origins. Dermatologica. 183: 31-35, 1991.
7. Monstrey S, Beele H, Kettler M, et al. Allogeneic cultured keratinocytes vs. cadaveric skin to cover wide-mesh autogenous split-thickness skin grafts. Ann. Plast. Surg. 43: 268-272, 1999.
8. Fritz D A. Burns & smoke inhalation. In: Stone C K, Humphries R L, eds. Current diagnosis & treatment: emergency medicine. 6^{th} ed. New York: McGraw-Hill, 836-848, 2008.
9. Biswas A, Bharara M, Hurst C, Armstrong D G, Rilo H, J. Diabetes Sci. Technol. 4, 808-819, 2010.
10. Xie J, Liu W, MacEwan M R, Yeh Y C, Thomopoulos S, Xia Y. Nanofiber membranes with controllable microwells and structural cues and their use in forming cell microarrays and neuronal networks, Small 7: 293-297, 2011.
11. Xie J, MacEwan M R, Ray W Z, Liu W, Siewe D Y, Xia Y. Radially-aligned electrospun nanofibers as dural substitutes for wound closure and tissue regeneration applications, ACS Nano 4: 5027-5036, 2010.
12. MacEwan M R, Xie J, Ray W Z, Xia Y, Nanofiber dural substitute and nanofiber dural patch. U.S. Patent Application Ser. No. 61/35,572
13. Orgill, D. P. Excision and skin grafting of thermal burns. N. Engl. J. Med. 2009, 360, 893-901.
14. Finkelstein, E. A.; Corso, P. S.; Miller, T. R. Associates. Incidence and economic burden of injuries in the United States. New York: Oxford University Press, 2006.
15. Falanga, V.; Faria, K.; Rober, L.; Joseph, V. Bioengineered skin constructs. Principles of Tissue Engineering. 3^{rd} ed. Burlington: Academic Press. 2007, 1167-1185.
16. Bishara, S. A.; Michel, C. Cultured epithelial autograft (CEA) in burn treatment: three decades later. Burns 2007, 33, 405-413.
17. Chester, D. L.; Balderson, D. S.; Papini, R. P. G. A review of keratinocyte delivery to the wound bed. J. Burn Care Rehab. 2004, 25, 266-275.
18. Williamson, J. S.; Snelling, C. F. T.; Clugston, P.; Macdonald, I. B.; Germann, E. Cultured epithelial autograft: five years of clinical experience with twenty-eight patients. J. Trauma 1995, 39, 309-319.
19. Green, H. Cultured cells for the treatment of disease. Sci. Am. 1991, 265, 96-102.
20. Ronfard, V.; Rives, J. M.; Neveux, Y.; Carsin, H.; Barrandon, Y. Long-term regeneration of human epidermis on third degree burns transplanted with autologous cultured epithelium grown on a fibrin matrix. Transplantation 2000, 70, 1588-1598.
21. Odessey, R. Addendum: multicenter experience with cultured epidermal autograft for treatment of burns. J. Burn Care Rehabil. 1992, 13, 174-180.
22. Leon-Villapalos, J.; Eldardiri, M.; Dziewulski, P. The use of human deceased donor skin allograft in burn care. Cell Tissue Bank 2010, 11, 99-104
23. Ong, Y. S.; Samuel, M.; Song, C. Meta-analysis of early excision of burns. Burns 2006, 32, 145-150.
24. Sophie, B. H.; Thomas, B.; Ernst, R. Tissue engineering of skin. Burns 2010, 36, 450-460.
25. Chen, X. L.; Liang, X.; Sun, L.; Wang, F.; Liu, S.; Wang, Y. J. Microskin autografting in the treatment of burns over 70% of total body surface area: 14 years of clinical experience. Burns 2011, 37, 973-980.
26. Lumenta, D. B.; Kamolz, L. P.; Frey, M. Adult burn patients with more than 60% TBSA involved-Meek and other techniques to overcome restricted skin harvest availability—the Viennese Concept. J. Burn Care Res. 2009, 30, 231-242.
27. Aiyer, P. M.; Gower, J. P. The mesh skin graft is very useful in the resurfacing of large burns. Burns 1999, 25, 467.
28. Kreis, R. W.; Mackie, D. P.; Hermans, R. R.; Vloemans, A. R. Expansion techniques for skin grafts: comparison between mesh and Meek island (sandwich-) grafts. Burns 1994, 20 Suppl 1, S39-42.
29. Zhang, M. L.; Chang, Z. D.; Han, X.; Zhu, M. Microskin grafting. I. Animal experiments. Burns Incl. Therm. Inj. 1986, 12, 540-543.
30. Zhang, M. L.; Wang, C. Y.; Chang, Z. D.; Cao, D. X.; Han, X. Microskin grafting. II. Clinical report. Burns Incl. Therm. Inj. 1986, 12, 544-548.
31. Zhang, M. L.; Chang, Z. D.; Wang, C. Y.; Fang, C. H. Microskin grafting in the treatment of extensive burns: a preliminary report. J. Trauma 1988, 28, 804-807.
32. Xie, J.; Liu, W.; MacEwan, M. R.; Yeh, Y. C.; Thomopoulos, S.; Xia, Y. Nanofiber membranes with controllable microwells and structural cues and their use in forming cell microarrays and neuronal networks. Small 2011, 7, 293-297.
33. Xie, J.; MacEwan, M. R.; Ray, W. Z.; Liu, W.; Siewe, D. Y.; Xia, Y. Radially aligned, electrospun nanofibers as dural substitutes for wound closure and tissue regeneration applications. ACS Nano 2010, 4, 5027-5036.
34. Gu, Q.; Xing, J. Z.; Huang, M.; He, C.; Chen, J. SN-38 loaded polymeric micelles to enhance cancer therapy. Nanotechnology 2012, 23, 205101.
35. Pastorino, L.; Pioli, F.; Zilli, M.; Converti, A.; Nicolini, C. Lipase-catalyzed degradation of poly(ε-caprolactone). Enzyme and Microbial Technology 2004, 35, 321-326.
36. Kweon, H. Y.; Yoo, M. K.; Park, I. K.; Kim, T. H.; Lee, H. C.; Lee, H. S.; Oh, J. S.; Akaike, T.; Cho, C. S. A novel degradable polycaprolactone networks for tissue engineering. Biomaterials 2003, 24, 801-808.
37. Lin, T. W. The algebraic view-point in microskin grafting in burned patients. Burns 1994, 20, 347-350.
38. Prabhakaran, M. P.; Venugopal, J.; Chan, C. K.; Ramakrishna, S. Surface modified electrospun nanofibrous scaffolds for nerve tissue engineering. Nanotechnology 2008, 19, 455102.
39. Martins, A.; Pinho, E. D.; Faria, S.; Pashkuleva, I.; Marques, A. P.; Reis, R. L.; Neves, N. M. Surface modification of electrospun polycaprolactone nanofiber meshes by plasma treatment to enhance biological performance. Small 2009, 5, 1195-1206.
40. Zhong, S. P.; Zhang, Y. Z; Lim, C. T. Tissue scaffolds for skin wound healing and dermal reconstruction. Wiley Interdiscip. Rev. Nanomed. Nanobiotechnol. 2010, 2, 510-525.
41. Kumbar, S. G.; James, R.; Nukavarapu, S. P.; Laurencin, C. T. Electrospun nanofiber scaffolds: engineering soft tissues. Biomed. Mater. 2008, 3, 034002.
42. Jin, G.; Prabhakaran, M. P.; Ramakrishna, S. Stem cell differentiation to epidermal lineages on electrospun substrates for skin tissue engineering. Acta Biomater. 2011, 7, 3113-3122.
43. Yang, Y.; Xia, T.; Zhi, W.; Wei, L.; Weng, J.; Zhang, C.; Li, X. Promotion of skin regeneration in diabetic rats by electropsun core-sheath fibers loaded with basic fibroblast growth factor. Biomaterials 2011, 32, 4243-4254.
44. Unnithan, A. R.; Barakatt, N. A.; Tirupathi, Pichiah, P. B.; Gnanasekaran, G.; Nirmala, R.; Cha, Y. S.; Jung, C. H.; EI-Newehy, M.; Kim, H. Y. Wound-dressing materials with antibacterial activity from electrospun polyurethane-dextran nanofiber mats containing ciprofloxacin HCl. Carbohydr. Polym. 2012, 90, 1786-1793.
45. Chen, J. P.; Chiang, Y. Bioactive electrospun silver nanoparticles-containing polyurethane nanofibers as wound dressings. J. Nanosci. Nanotechnol. 2010, 10, 7560-75644.
46. Chandrasekaran, A. R.; Venugopal, J.; Sundarrajan, S.; Ramakrishna, S. Fabrication of a nanofibrous scaffold with improved bioactivity for culture of human dermal fibroblasts for skin regeneration. Biomed. Mater. 2011, 6, 015001.
47. Gumusderelioglu, M.; Dalkiranoglu, S.; Aydin, R. S.; Cakmak, S. A novel dermal substitute based on biofunctionalized electrospun PCL nanofibrous matrix. J. Biomed. Mater. Res. A. 2011, 98, 461-472.
48. Tchemtchoua, V. T.; Atanasova, G.; Agil, A.; Filee, P.; Garbacki, N.; Vanhooteghem, O.; Deroanne, C.; Noel, A.; Jerome, C.; Nusgens, B.; Poumay, Y.; Colige, A. Development of a chitosan nanofibrillar scaffold for skin repair and regeneration. Biomarcromolecules 2011, 12, 3194-3204.
49. Schnell, E.; Klinkhammer, K.; Balzer, S.; Brook, G.; Klee, D.; Dalton, P.; Mey, J. Guidance of glial cell migration and axonal growth on electrospun nanofibers of poly-epsilon-caprolactone and a collagen/poly-epsilon-caprolactone blend. Biomaterials 2007, 28, 3012-3025.
50. Corey, J. M.; Lin, D. Y.; Mycek, K. B.; Chen, Q.; Samuel, S.; Feldman, E. L.; Martin, D. C. Aligned electrospun nanofibers specify the direction of dorsal root ganglia neurite growth. J. Biomed. Mater. Res. A. 2007, 83, 636-645.
51. Schnell, E.; Klinkhammer, K.; Balzer, S.; Brook, G.; Klee, D.; Dalton, P.; Mey, J. Guidance of glial cell migration and axonal growth on electrospun nanofibers of poly-epsilon-caprolactone and a collagen/poly-epsilon-caprolactone blend. Biomaterials 2007, 28, 3012-3025.
52. Lari, A. R.; Gang, R. K. Expansion technique for skin grafts (MEEK technique) in the treatment of severely burned patients. Burns 2001, 27, 61-66.
53. Hsieh, C. S.; Schuong, J. Y.; Huang, W. S.; Huang, T. T. Five years' experience of the modified Meek technique in the management of extensive burns. Burns 2008, 34, 350-354.
54. Ma, D.; Liu, M.; Ma, B.; Wang, X. J.; Pen, Y.; Zhang, P.; Li, C. Y.; Liu, Y. M.; Wen, K.; Xu, X. F.; Zhan, H.; Zhang, W.; Liu, Y. L. A novel method for wound healing—skin suspension accelerates donor site wound healing. Wounds 2011, 23, 107-110.
55. Converse, J. M.; Smahel, J.; Ballentyne, D. L. Inosculation of vessels of skin graft and host bed: a fortuitous encounter. Br. J. Plast. Surg. 1975, 28, 274-282.
56. Bush, K. A.; Pins, G. D. Development of microfabricated dermal epidermal regenerative matrices to evaluate the role of cellular microenvironments on epidermal morphogenesis, Tissue Eng. A 2012, doi: 10.1089/ten.tea.2011.0479.
57. Odland, G. The morphology of the attachment between the dermis and the epidermis, Anat. Record. 1950, 108, 399-413.
58. Fawcet, D. W.; Jensh, R. P. Concise Histology. New York, N.Y.: Chapmen and Hall, 1997.
59. Hierner, R.; Degreef, H.; Vranckx, J. J.; Garmyn, M.; Massage, P.; van Brussel, M Skin grafting and wound healing-the ‘dermato-plastic team approach’. Clin. Dermatol. 2005, 23, 343-352.
60. Johan, D. H.; Margareta, F. N.; Gunnar, K.; Gunther, W.; Ole, S.; Niels, B.; Mona, S. B. The cathelicidin anti-microbial peptide IL-37 is involved in re-epithelialization of human skin wounds and is lacking in chronic ulcer epithelium. J. Invest. Dermatol. 2003, 120, 379-389.
61. Bottcher-Haberzeth, S.; Biedermann, T.; Reichmann, E. Tissue engineering of skin. Burns 2010, 36, 450-460.
62. Orgill, D.; Butler, C.; Regan, J.; Barlow, M.; Yanna, I. V.; Compton, C. C. Vascularized collagen-glycosaminoglycan matrix provides a dermal substrate and improves take of cultures epithelial autografts. Plast. Reconstr. Surg. 1998, 102, 423-429.
63. Eldardiri, M.; Martin, Y.; Roxburgh, J.; Lawrence-Watt, D. J.; Sharpe, J. R. Wound contraction is significantly reduced by the use of microcarriers to deliver keratinocytes and fibroblasts in an in vivo pig model of wound repair and regeneration. Tissue Eng. Part A. 2012, 18, 587-597.
64. Martin, P. Wound healing—aiming for perfect skin regeneration. Science 1997, 276, 75-81.
65. Singer, A. J.; Clark, R. A. Cutaneous wound healing. N. Engl. J. Med. 1999, 341, 738-746.
66. National Research Council. Guide for the Care and Use of Laboratory Animals. Washington, D.C.: National Academy Press, 1985; NIH publication no. 86-23.
67. Li, W. W.; Guo, T. Z.; Li, X. Q.; Kingery, W. S.; Clark, J. D. Fracture induces keratinocyte activation, proliferation, and expression of pro-nociceptive inflammatory mediators. Pain 2010, 151, 843-852.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A composition, comprising:
a first nanofiber scaffold including microwells configured to be seeded with one or more relevant cells, a skin tissue, or combinations thereof; and
a second nanofiber scaffold layered on the first nanofiber scaffold.

2. The composition of claim 1, wherein the relevant cells are selected from the group consisting of adult stem cells, embryonic stem cells, induced pluripotent cells, primary cells, and combinations thereof.

3. The composition of claim 2, wherein the adult stem cells are adipose-derived stem cells.

4. The composition of wherein the primary cells are skin cells.

5. The composition of claim 1, wherein the skin tissue is minced skin tissue.

6. The composition of claim 5, wherein each piece of the minced skin tissue is about 0.1 mm to about 1 mm in diameter.

7. The composition of claim 1, wherein the first nanofiber scaffold comprises uniaxially-aligned nanofibers between the microwells and random nanofibers on the microwells.

8. The composition of claim 1, wherein the second nanofiber scaffold comprises radially-aligned nanofibers.

9. The composition of claim 1, wherein the first nanofiber scaffold, the second nanofiber scaffold, or both are comprised of a biodegradable polymer.

10. The composition of claim 9, wherein the biodegradable polymer is selected from the group consisting of synthetic polymers, natural polymers, inorganic materials, and combinations thereof.

11. The composition of claim 9, wherein the biodegradable polymer is comprised of polycaprolactone.

12. The composition of claim 1, further comprising an extracellular matrix protein.

13. The composition of claim 12, wherein the extracellular matrix protein is selected from fibronectin, laminin, collagen, or a combination thereof.

14. The composition of claim 1, further comprising a growth factor.

15. The composition of claim 14, wherein the growth factor is selected from the group consisting of vascular endothelial growth factor (VEGF), basic fibroblast growth factor (bFGF), insulin-like growth factor (IGF), placental growth factor (PIGF), Angl, platelet derived growth factor-BB (PDGF-BB), transforming growth factor β (TGF-β), human epidermal growth factor (hEGF), keratinocyte growth factor, and combinations thereof.

16. The composition of claim 1, further comprising a therapeutic agent.

17. The composition of claim 16, wherein the therapeutic agent is an anti-inflammatory agent, an antibiotic, or a combination thereof.

18. The composition of claim 1, wherein the microwells have a diameter of about 0.1 mm to about 10 mm.

19. The composition of claim 1, wherein the microwells have a depth of about 20 μm to about 2 mm.

20. The composition of claim 1, wherein the microwells are arranged in a square array, a hexagonal array, or a combination thereof.

21. A method for treating damaged skin in a subject, comprising:
providing a composition comprising:
a first nanofiber scaffold including microwells seeded with one or more relevant cells, a skin tissue, or combinations thereof, and
a second nanofiber scaffold layered on the first nanofiber scaffold; and
applying an effective amount of the composition to a site of damaged skin on the subject.

22. The method of claim 21, wherein the relevant cells are selected from the group consisting of adult stem cells, embryonic stem cells, induced pluripotent cells, primary cells, and combinations thereof.

23. The method of claim 21, wherein the first nanofiber scaffold comprises uniaxially-aligned nanofibers between the microwells and random nanofibers on the microwells.

24. The method of claim 21, wherein the second nanofiber scaffold comprises radially-aligned nanofibers.

25. The method of claim 21, wherein the composition further comprises a growth factor, an extracellular matrix protein, a therapeutic agent, or a combination thereof.

26. The method of claim 21, wherein the first nanofiber scaffold, the second nanofiber scaffold, or both are comprised of a biodegradable polymer.

27. The method of claim 21, wherein applying an effective amount of the composition comprises covering at least the damaged skin with the composition.

28. A method for making a nanofiber scaffold composition, comprising:
electrospinning a first biodegradable polymer onto a first collector comprising beads to create a first nanofiber scaffold including microwells configured to be seeded with one or more relevant cells, a skin tissue, or combinations thereof;
electrospinning a second biodegradable polymer onto a second collector comprising a ring electrode and a point electrode to create a second nanofiber scaffold;
seeding the one or more relevant cells, the skin tissue, or combinations thereof in the microwells of the first nanofiber scaffold; and
layering the second nanofiber scaffold on the first nanofiber scaffold.

29. The method of claim 28, wherein the step of seeding the one or more relevant cells comprises loading a solution of the one or more relevant cells into the microwells of the first nanofiber scaffold.

30. The method of claim 28, wherein the relevant cells are selected from the group consisting of adult stem cells, embryonic stem cells, induced pluripotent cells, primary cells, and combinations thereof.

31. The method of claim 28, wherein the step of seeding the skin tissue further comprises mincing the skin tissue and loading the minced skin tissue into the microwells of the first nanofiber scaffold.

32. The method of claim 28, wherein the first biodegradable polymer and the second biodegradable polymer are selected from the group consisting of synthetic polymers, natural polymers, inorganic materials, and combinations thereof.

33. The method of claim 28, wherein the first biodegradable polymer, the second biodegradable polymer, or both are comprised of polycaprolactone.

34. The method of claim 28, further comprising a step of attaching an extracellular matrix protein to the first nanofiber scaffold, the second nanofiber scaffold, or both.

35. The method of claim 28, further comprising a step of attaching a growth factor to the first nanofiber scaffold, the second nanofiber scaffold, or both.

36. The method of claim 28, further comprising a step of attaching a therapeutic agent to the first nanofiber scaffold, the second nanofiber scaffold, or both.

37. The method of claim 28, wherein the beads are arranged in a square array, a hexagonal array, or combinations thereof.

38. The method of claim 28, wherein the first nanofiber scaffold comprises uniaxially-aligned nanofibers between microwells and randomly-aligned nanofibers on microwells, and wherein the second nanofiber scaffold comprises radially-aligned nanofibers.
